SEQUENCE LISTING (99 sequences)

SEQ ID NO: 1 - DNA, 830 bp, synthetic construct
  misc_feature 1..830: note = Beta AS1 (T87Q) (not modified)
  source 1..830: mol_type = other DNA
ttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcaggtgcacca

SEQ ID NO: 2 - DNA, 831 bp, synthetic construct
  misc_feature 1..831: note = Beta AS3 (not modified)
  source 1..831: mol_type = other DNA
ttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccacagggcagtaacggcagacttctcctcaggagtcaggtgcaccat

SEQ ID NO: 3 - DNA, 848 bp, synthetic construct
  misc_feature 1..848: note = Gamma-beta hybrid
  source 1..848: mol_type = other DNA
tcagtggtatctggaggacagggcactggccactgcagtcaccatcttctgccaggaagcctgcacctcaggggtgaattctttgccgaaatggattgccaaaacggtcaccagcacatttcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcacgggtcccatagactcaccttgaagttctcaggatccacatgcagcttgtcacagtgcagttcactcagctgggcaaaggtgcccttgagatcatccaggtgctttgtggcatctcccaaggaagtcagcaccttcttgccatgtgccttgactttggggttgcccatgatggcagaggcagaggacaggttgccaaagctgtcaaagaacctctgggtccatgggtagacaaccaggagcctaagggtgggaaaatagaccaataggcagagagagtcagtgcctatcagaaacccaagagtcttctctgtctccacatgcccagtttctattggtctccttaaacctgtcttgtaaccttgataccaaccttcccagggtttctcctccagcatcttccacattcaccttgccccacaggcttgtgatagtagccttgtcctcctctgtgaaatgacccat

SEQ ID NO: 4 - DNA, 848 bp, synthetic construct
  misc_feature 1..848: note = Gamma-beta hybrid AS2 (G16D and D22A)
  source 1..848: mol_type = other DNA
tcagtggtatctggaggacagggcactggccactgcagtcaccatcttctgccaggaagcctgcacctcaggggtgaattctttgccgaaatggattgccaaaacggtcaccagcacatttcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcacgggtcccatagactcaccttgaagttctcaggatccacatgcagcttgtcacagtgcagttcactcagctgggcaaaggtgcccttgagatcatccaggtgctttgtggcatctcccaaggaagtcagcaccttcttgccatgtgccttgactttggggttgcccatgatggcagaggcagaggacaggttgccaaagctgtcaaagaacctctgggtccatgggtagacaaccaggagcctaagggtgggaaaatagaccaataggcagagagagtcagtgcctatcagaaacccaagagtcttctctgtctccacatgcccagtttctattggtctccttaaacctgtcttgtaaccttgataccaaccttcccagggtttctcctccagcggcttccacattcaccttgtcccacaggcttgtgatagtagccttgtcctcctctgtgaaatgacccat

SEQ ID NO: 5 - DNA, 831 bp, synthetic construct
  misc_feature 1..831: note = Delta-beta hybrid
  source 1..831: mol_type = other DNA
tcaatggtacttgtgagccagggcattagccacaccagccaccaccttctgataggcagcctgcatttgtggggtgaattccttgccaaagttgcgggccagcacacacaccagcacattgcccaagagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgagaaaaagtgcccttgaggttgtccaggtgagccaggccatcactaaaggcacctagcaccttcttgccatgagccttcaccttagggttgcccataacagcatcaggagaggacagatccccaaaggactcaaagaacctctgggtccaagggtagaccaccagtaatctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaactgcatccacgttcactttgccccacagggcattgacagcagtcttctcctcaggagtcagatgcaccat

SEQ ID NO: 6 - DNA, 848 bp, synthetic construct
  misc_feature 1..848: note = Delta-beta hybrid AS1 (G16D)
  source 1..848: mol_type = other DNA
tcaatggtacttgtgagccagggcattagccacaccagccaccaccttctgataggcagcctgcatttgtggggtgaattccttgccaaagttgcgggccagcacacacaccagcacattgcccaagagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcacgggtcccatagactcaccctgaagttctcaggatccacgtgcagcttgtcacagtgcagctcactcagctgagaaaaagtgcccttgaggttgtccaggtgagccaggccatcactaaaggcacctagcaccttcttgccatgagccttcaccttagggttgcccataacagcatcaggagaggacagatccccaaaggactcaaagaacctctgggtccaagggtagaccaccagtaatctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaactgcatccacgttcactttgtcccacagggcattgacagcagtcttctcctcaggagtcagatgcaccat

SEQ ID NO: 7 - DNA, 831 bp, synthetic construct
  misc_feature 1..831: note = Beta AS1 (T87Q) (modified to avoid targeting by gRNA D)
  source 1..831: mol_type = other DNA
ttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccagagagcggtcacagcggacttctcctcaggagtcaggtgcaccat

SEQ ID NO: 8 - DNA, 831 bp, synthetic construct
  misc_feature 1..831: note = Beta AS3 (modified to avoid targeting by gRNA D)
  source 1..831: mol_type = other DNA
ttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccat

SEQ ID NO: 9 - DNA, 20 bp, synthetic construct
  misc_feature 1..20: note = Primer HBBex1 F
  source 1..20: mol_type = other DNA
cagcatcaggagtggacaga

SEQ ID NO: 10 - DNA, 20 bp, synthetic construct
  misc_feature 1..20: note = Primer HBBex1 R
  source 1..20: mol_type = other DNA
agtcagggcagagccatcta

SEQ ID NO: 11 - DNA, 22 bp, synthetic construct
  misc_feature 1..22: note = Primer HBB F
  source 1..22: mol_type = other DNA
gcaaggtgaacgtggatgaagt

SEQ ID NO: 12 - DNA, 23 bp, synthetic construct
  misc_feature 1..23: note = Primer HBB R
  source 1..23: mol_type = other DNA
taacagcatcaggagtggacaga

SEQ ID NO: 13 - DNA, 21 bp, synthetic construct
  misc_feature 1..21: note = Primer HBA1 F
  source 1..21: mol_type = other DNA
cggtcaacttcaagctcctaa

SEQ ID NO: 14 - DNA, 20 bp, synthetic construct
  misc_feature 1..20: note = Primer HBA1 R
  source 1..20: mol_type = other DNA
acagaagccaggaacttgtc

SEQ ID NO: 15 - DNA, 24 bp, synthetic construct
  misc_feature 1..24: note = Oligo FOR-Opt_gRNA B
  source 1..24: mol_type = other DNA
caccgtaacggcagacttctcctc

SEQ ID NO: 16 - DNA, 24 bp, synthetic construct
  misc_feature 1..24: note = Oligo REV-Opt_gRNA B
  source 1..24: mol_type = other DNA
aaacgaggagaagtctgccgttac

SEQ ID NO: 17 - DNA, 24 bp, synthetic construct
  misc_feature 1..24: note = Oligo FOR-Opt_gRNA D
  source 1..24: mol_type = other DNA
caccgtctgccgttactgccctgt

SEQ ID NO: 18 - DNA, 24 bp, synthetic construct
  misc_feature 1..24: note = Oligo REV-Opt_gRNA D
  source 1..24: mol_type = other DNA
aaacacagggcagtaacggcagac

SEQ ID NO: 19 - DNA, 25 bp, synthetic construct
  misc_feature 1..25: note = Oligo FOR-Opt_gRNA E
  source 1..25: mol_type = other DNA
caccgaaggtgaacgtggatgaagt

SEQ ID NO: 20 - DNA, 25 bp, synthetic construct
  misc_feature 1..25: note = Oligo REV-Opt_gRNA E
  source 1..25: mol_type = other DNA
aaacacttcatccacgttcaccttc

SEQ ID NO: 21 - DNA, 18 bp, synthetic construct
  misc_feature 1..18: note = Primer HBB-AS3 F
  source 1..18: mol_type = other DNA
aagggcacctttgcccag

SEQ ID NO: 22 - DNA, 22 bp, synthetic construct
  misc_feature 1..22: note = Primer HBB-AS3 R
  source 1..22: mol_type = other DNA
gccaccactttctgataggcag

SEQ ID NO: 23 - DNA, 20 bp, synthetic construct
  misc_feature 1..20: note = gRNA A spacer
  source 1..20: mol_type = other DNA
cttgccccacagggcagtaa

SEQ ID NO: 24 - DNA, 20 bp, synthetic construct
  misc_feature 1..20: note = gRNA B spacer
  source 1..20: mol_type = other DNA
gtaacggcagacttctcctc

SEQ ID NO: 25 - DNA, 19 bp, synthetic construct
  misc_feature 1..19: note = gRNA D spacer
  source 1..19: mol_type = other DNA
tctgccgttactgccctgt

SEQ ID NO: 26 - DNA, 20 bp, synthetic construct
  misc_feature 1..20: note = gRNA E spacer
  source 1..20: mol_type = other DNA
aaggtgaacgtggatgaagt

SEQ ID NO: 27 - DNA, 20 bp, synthetic construct
  misc_feature 1..20: note = gRNA F spacer
  source 1..20: mol_type = other DNA
gtgtggcaaaggtgcccttg

SEQ ID NO: 28 - DNA, 20 bp, synthetic construct
  misc_feature 1..20: note = gRNA G spacer
  source 1..20: mol_type = other DNA
acagtgcagctcactcagtg

SEQ ID NO: 29 - DNA, 20 bp, synthetic construct
  misc_feature 1..20: note = gRNA H spacer
  source 1..20: mol_type = other DNA
tctgccgttactgccctgtg

SEQ ID NO: 30 - DNA, 20 bp, synthetic construct
  misc_feature 1..20: note = gRNA I spacer
  source 1..20: mol_type = other DNA
cgttactgccctgtggggca

SEQ ID NO: 31 - DNA, 20 bp, synthetic construct
  misc_feature 1..20: note = gRNA J spacer
  source 1..20: mol_type = other DNA
gtctgccgttactgccctgt

SEQ ID NO: 32 - DNA, 20 bp, synthetic construct
  misc_feature 1..20: note = gRNA K spacer
  source 1..20: mol_type = other DNA
cagctcactcagtgtggcaa

SEQ ID NO: 33 - DNA, 20 bp, synthetic construct
  misc_feature 1..20: note = gRNA L spacer
  source 1..20: mol_type = other DNA
tcccacccttaggctgctgg

SEQ ID NO: 34 - DNA, 20 bp, synthetic construct
  misc_feature 1..20: note = gRNA M spacer
  source 1..20: mol_type = other DNA
ggctgctggtggtctaccct

SEQ ID NO: 35 - DNA, 20 bp, synthetic construct
  misc_feature 1..20: note = gRNA N spacer
  source 1..20: mol_type = other DNA
ccccacagggcagtaacggc

SEQ ID NO: 36 - DNA, 20 bp, synthetic construct
  misc_feature 1..20: note = gRNA O spacer
  source 1..20: mol_type = other DNA
taacggcagacttctccaca

SEQ ID NO: 37 - DNA, 27 bp, synthetic construct
  misc_feature 1..27: note = Oligo FOR-gRNA A
  source 1..27: mol_type = other DNA
acaccgcttgccccacagggcagtaag

SEQ ID NO: 38 - DNA, 27 bp, synthetic construct
  misc_feature 1..27: note = Oligo REV-gRNA A
  source 1..27: mol_type = other DNA
aaaacttactgccctgtggggcaagcg

SEQ ID NO: 39 - DNA, 26 bp, synthetic construct
  misc_feature 1..26: note = Oligo FOR-gRNA B
  source 1..26: mol_type = other DNA
acaccgtaacggcagacttctcctcg

SEQ ID NO: 40 - DNA, 26 bp, synthetic construct
  misc_feature 1..26: note = Oligo REV-gRNA B
  source 1..26: mol_type = other DNA
aaaacgaggagaagtctgccgttacg

SEQ ID NO: 41 - DNA, 26 bp, synthetic construct
  misc_feature 1..26: note = Oligo FOR-gRNA D
  source 1..26: mol_type = other DNA
acaccgtctgccgttactgccctgtg

SEQ ID NO: 42 - DNA, 26 bp, synthetic construct
  misc_feature 1..26: note = Oligo REV-gRNA D
  source 1..26: mol_type = other DNA
aaaacacagggcagtaacggcagacg

SEQ ID NO: 43 - DNA, 27 bp, synthetic construct
  misc_feature 1..27: note = Oligo FOR-gRNA E
  source 1..27: mol_type = other DNA
acaccgaaggtgaacgtggatgaagtg

SEQ ID NO: 44 - DNA, 27 bp, synthetic construct
  misc_feature 1..27: note = Oligo REV-gRNA E
  source 1..27: mol_type = other DNA
aaaacacttcatccacgttcaccttcg

SEQ ID NO: 45 - DNA, 10195 bp, synthetic construct
  misc_feature 1..10195: note = LV.GLOBE.betaAS3-GLOBIN
  source 1..10195: mol_type = other DNA
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccaaatggcggccgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 46 - DNA, 10195 bp, synthetic construct
  misc_feature 1..10195: note = LV.GLOBE.betaAS3-GLOBIN Sal1 (with restriction site for Sal1)
  source 1..10195: mol_type = other DNA
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccaaatggcggccgtacctttaagaccaatgacttacaaggcagctgtcgaccttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 47 - DNA, 10578 bp, synthetic construct
  misc_feature 1..10578: note = LV.GLOBE.betaAS3-GLOBIN.gRNAD-OPTIMIZED (harboring the expression cassette for grna D - dang scaffold - U6 promoter)
  source 1..10578: mol_type = other DNA
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccaaatggcggccgtacctttaagaccaatgacttacaaggcagctgtcgacaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtctgccgttactgccctgtggtttcagagctatgctggaaacagcatagcaagttgaaataaggctagtccgttatcaacttgaaaaagtggcaccgagtcggtgctttttttgtcgaccttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 48 - DNA, 18 bp, synthetic construct
  misc_feature 1..18: note = gRNA against gamma-delta intergenic region
  source 1..18: mol_type = other DNA
ggtgctctatacttccta

SEQ ID NO: 49 - DNA, 20 bp, synthetic construct
  misc_feature 1..20: note = gRNA A spacer inverted sequence
  source 1..20: mol_type = other DNA
gaacggggtgtcccgtcatt

SEQ ID NO: 50 - DNA, 20 bp, synthetic construct
  misc_feature 1..20: note = HBG genes inverted sequence OFF-TARGET gRNA A
  source 1..20: mol_type = other DNA
gaacggggtgtccgaacact

SEQ ID NO: 51 - DNA, 20 bp, synthetic construct
  misc_feature 1..20: note = HBD gene inverted sequence OFF-TARGET gRNA A
  source 1..20: mol_type = other DNA
aaacggggtgtcccgtaact

SEQ ID NO: 52 - DNA, 20 bp, synthetic construct
  misc_feature 1..20: note = gRNA B spacer inverted sequence
  source 1..20: mol_type = other DNA
cattgccgtctgaagaggag

SEQ ID NO: 53 - DNA, 20 bp, synthetic construct
  misc_feature 1..20: note = HBG genes inverted sequence OFF-TARGET gRNA B
  source 1..20: mol_type = other DNA
tatcatcggaacaggaggag

SEQ ID NO: 54 - DNA, 20 bp, synthetic construct
  misc_feature 1..20: note = HBD gene inverted sequence OFF-TARGET gRNA B
  source 1..20: mol_type = other DNA
aactgtcgtcagaagaggag

SEQ ID NO: 55 - DNA, 19 bp, synthetic construct
  misc_feature 1..19: note = gRNA D spacer inverted sequence
  source 1..19: mol_type = other DNA
agacggcaatgacgggaca

SEQ ID NO: 56 - DNA, 19 bp, synthetic construct
  misc_feature 1..19: note = HBG genes inverted sequence OFF-TARGET gRNA D
  source 1..19: mol_type = other DNA
cgatgatagtgttcggaca

SEQ ID NO: 57 - DNA, 19 bp, synthetic construct
  misc_feature 1..19: note = HBD gene inverted sequence OFF-TARGET gRNA D
  source 1..19: mol_type = other DNA
tgacgacagttacgggaca

SEQ ID NO: 58 - DNA, 20 bp, synthetic construct
  misc_feature 1..20: note = gRNA E spacer inverted sequence
  source 1..20: mol_type = other DNA
ttccacttgcacctacttca

SEQ ID NO: 59 - DNA, 20 bp, synthetic construct
  misc_feature 1..20: note = HBG genes inverted sequence OFF-TARGET gRNA E
  source 1..20: mol_type = other DNA
ttccacttacaccttctacg

SEQ ID NO: 60 - DNA, 20 bp, synthetic construct
  misc_feature 1..20: note = HBD gene inverted sequence OFF-TARGET gRNA E
  source 1..20: mol_type = other DNA
tttcacttgcacctacgtca

SEQ ID NO: 61 - RNA, 101 nt, synthetic construct
  misc_feature 1..101: note = ORIGINAL gRNA SCAFFOLD sequence
  misc_difference 1..20: note = n is a, c, g, or u
  source 1..101: mol_type = other RNA
nnnnnnnnnnnnnnnnnnnngttttagagctagaaatagcaagttaaaataaggctagtccgttatcaacttgaaaaagtggcaccgagtcggtgcttttt

SEQ ID NO: 62 - RNA, 112 nt, synthetic construct
  misc_feature 1..112: note = OPTIMIZED gRNA SCAFFOLD sequence
  misc_difference 1..20: note = n is a, c, g, or u
  source 1..112: mol_type = other RNA
nnnnnnnnnnnnnnnnnnnngtttcagagctatgctggaaacagcatagcaagttgaaataaggctagtccgttatcaacttgaaaaagtggcaccgagtcggtgctttttt

SEQ ID NO: 63 - RNA, 101 nt, synthetic construct
  misc_feature 1..101: note = ORIGINAL SCAFFOLD gRNA B sequence
  source 1..101: mol_type = other RNA
gtaacggcagacttctcctcgttttagagctagaaatagcaagttaaaataaggctagtccgttatcaacttgaaaaagtggcaccgagtcggtgcttttt

SEQ ID NO: 64 - RNA, 100 nt, synthetic construct
  misc_feature 1..100: note = ORIGINAL SCAFFOLD gRNA D sequence
  source 1..100: mol_type = other RNA
tctgccgttactgccctgtgttttagagctagaaatagcaagttaaaataaggctagtccgttatcaacttgaaaaagtggcaccgagtcggtgcttttt

SEQ ID NO: 65 - RNA, 101 nt, synthetic construct
  misc_feature 1..101: note = ORIGINAL SCAFFOLD gRNA E sequence
  source 1..101: mol_type = other RNA
aaggtgaacgtggatgaagtgttttagagctagaaatagcaagttaaaataaggctagtccgttatcaacttgaaaaagtggcaccgagtcggtgcttttt

SEQ ID NO: 66 - RNA, 113 nt, synthetic construct
  misc_feature 1..113: note = OPTIMIZED SCAFFOLD gRNA B sequence
  source 1..113: mol_type = other RNA
gtaacggcagacttctcctcggtttcagagctatgctggaaacagcatagcaagttgaaataaggctagtccgttatcaacttgaaaaagtggcaccgagtcggtgctttttt

SEQ ID NO: 67 - RNA, 112 nt, synthetic construct
  misc_feature 1..112: note = OPTIMIZED SCAFFOLD gRNA D sequence
  source 1..112: mol_type = other RNA
tctgccgttactgccctgtggtttcagagctatgctggaaacagcatagcaagttgaaataaggctagtccgttatcaacttgaaaaagtggcaccgagtcggtgctttttt

SEQ ID NO: 68 - RNA, 114 nt, synthetic construct
  misc_feature 1..114: note = OPTIMIZED SCAFFOLD gRNA E sequence
  source 1..114: mol_type = other RNA
gaaggtgaacgtggatgaagtggtttcagagctatgctggaaacagcatagcaagttgaaataaggctagtccgttatcaacttgaaaaagtggcaccgagtcggtgctttttt

SEQ ID NO: 69 - DNA, 34 bp, synthetic construct
  misc_feature 1..34: note = HBB gene SCD sequence
  source 1..34: mol_type = other DNA
cctgtggagaagtctgccgttactgccctgtggg

SEQ ID NO: 70 - protein, 11 aa, synthetic construct
  REGION 1..11: note = Beta-S globin protein sequence
PVEKSAVTALW

SEQ ID NO: 71 - DNA, 34 bp, synthetic construct
  misc_feature 1..34: note = HBB-AS3 modified sequence
  source 1..34: mol_type = other DNA
cctgaggagaagtccgctgtgaccgctctctggg

SEQ ID NO: 72 - protein, 11 aa, synthetic construct
  REGION 1..11: note = Beta-AS3 globin sequence
PEEKSAVTALW

SEQ ID NO: 73 - DNA, 20 bp, synthetic construct
  misc_feature 1..20: note = gRNA 13bp-del spacer
  source 1..20: mol_type = other DNA
cttgtcaaggctattggtca

SEQ ID NO: 74 - DNA, 20 bp, synthetic construct
  misc_feature 1..20: note = gRNA BCL11A spacer
  source 1..20: mol_type = other DNA
cacaggctccaggaagggtt

SEQ ID NO: 75 - DNA, 10584 bp, synthetic construct
  misc_feature 1..10584: note = LV.GLOBE-AS3modified.gRNA-BCL11Aenhancer
  source 1..10584: mol_type = other DNA
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccgtcgacaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcacaggctccaggaagggttgtttcagagctatgctggaaacagcatagcaagttgaaataaggctagtccgttatcaacttgaaaaagtggcaccgagtcggtgctttttttgtcgacactagttctagagcggccaaatggcggccgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 76 - DNA, 10584 bp, synthetic construct
  misc_feature 1..10584: note = LV.GLOBE-AS3modified.gRNA-13bp-del
  source 1..10584: mol_type = other DNA
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccgtcgacaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcttgtcaaggctattggtcagtttcagagctatgctggaaacagcatagcaagttgaaataaggctagtccgttatcaacttgaaaaagtggcaccgagtcggtgctttttttgtcgacactagttctagagcggccaaatggcggccgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 77 - DNA, 20 bp, synthetic construct
  misc_feature 1..20: note = BCL11A-TIDE FORWARD
  source 1..20: mol_type = other DNA
tggacagcccgacagatgaa

SEQ ID NO: 78 - DNA, 20 bp, synthetic construct
  misc_feature 1..20: note = BCL11A-TIDE REVERSE
  source 1..20: mol_type = other DNA
aaaagcgatacagggctggc

SEQ ID NO: 79 - DNA, 30 bp, synthetic construct
  misc_feature 1..30: note = 13bp-del-TIDE FORWARD
  source 1..30: mol_type = other DNA
aaaaacggctgacaaaagaagtcctggtat

SEQ ID NO: 80 - DNA, 30 bp, synthetic construct
  misc_feature 1..30: note = 13bp-del-TIDE REVERSE
  source 1..30: mol_type = other DNA
ataacctcagacgttccagaagcgagtgtg

SEQ ID NO: 81 - DNA, 19 bp, synthetic construct
  misc_feature 1..19: note = HBG1 + HBG2 FORWARD
  source 1..19: mol_type = other DNA
cctgtcctctgcctctgcc

SEQ ID NO: 82 - DNA, 19 bp, synthetic construct
  misc_feature 1..19: note = HBG1 + HBG2 REVERSE
  source 1..19: mol_type = other DNA
ggattgccaaaacggtcac

SEQ ID NO: 83 - DNA, 18 bp, synthetic construct
  misc_feature 1..18: note = HBB FORWARD
  source 1..18: mol_type = other DNA
aagggcacctttgccaca

SEQ ID NO: 84 - DNA, 22 bp, synthetic construct
  misc_feature 1..22: note = HBB REVERSE
  source 1..22: mol_type = other DNA
gccaccactttctgataggcag

SEQ ID NO: 85 - DNA, 19 bp, synthetic construct
  misc_feature 1..19: note = HBD FORWARD
  source 1..19: mol_type = other DNA
caagggcactttttctcag

SEQ ID NO: 86 - DNA, 19 bp, synthetic construct
  misc_feature 1..19: note = HBD REVERSE
  source 1..19: mol_type = other DNA
aattccttgccaaagttgc

SEQ ID NO: 87 - DNA, 20 bp, synthetic construct
  misc_feature 1..20: note = BCL11A FORWARD
  source 1..20: mol_type = other DNA
aaccccagcacttaagcaaa

SEQ ID NO: 88 - DNA, 20 bp, synthetic construct
  misc_feature 1..20: note = BCL11A REVERSE
  source 1..20: mol_type = other DNA
ggaggtcatgatccccttct

SEQ ID NO: 89 - DNA, 20 bp, synthetic construct
  misc_feature 1..20: note = BCL11AXL FORWARD
  source 1..20: mol_type = other DNA
atgcgagctgtgcaactatg

SEQ ID NO: 90 - DNA, 20 bp, synthetic construct
  misc_feature 1..20: note = BCL11AXL REVERSE
  source 1..20: mol_type = other DNA
gtaaacgtccttccccacct

SEQ ID NO: 91 - DNA, 26 bp, synthetic construct
  misc_feature 1..26: note = GAPDH FORWARD
  source 1..26: mol_type = other DNA
cttcattgacctcaactacatggttt

SEQ ID NO: 92 - DNA, 22 bp, synthetic construct
  misc_feature 1..22: note = GAPDH REVERSE
  source 1..22: mol_type = other DNA
tgggatttccattgatgacaag

SEQ ID NO: 93 - DNA, 10584 bp, synthetic construct
  misc_feature 1..10584: note = LV.GLOBE-AS3modified.gRNA-luciferase
  source 1..10584: mol_type = other DNA
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccgtcgacaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcttcgaaatgtccgttcggtgtttcagagctatgctggaaacagcatagcaagttgaaataaggctagtccgttatcaacttgaaaaagtggcaccgagtcggtgctttttttgtcgacactagttctagagcggccaaatggcggccgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 94 - DNA, 10583 bp, synthetic construct
  misc_feature 1..10583: note = LV.GLOBE-AS3modified.gRNAD
  source 1..10583: mol_type = other DNA
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccagagagcggtcacagcggacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccgtcgacaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtctgccgttactgccctgtgtttcagagctatgctggaaacagcatagcaagttgaaataaggctagtccgttatcaacttgaaaaagtggcaccgagtcggtgctttttttgtcgacactagttctagagcggccaaatggcggccgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 95 - protein, 31 aa, Homo sapiens
RGLAEGGVEDVNVKGWLATVASKEEPTLHVM

SEQ ID NO: 96 - DNA, 93 bp, Homo sapiens
  source 1..93: mol_type = unassigned DNA
ggacgggtcccggagtggtggttgaagtaggtgcaagtggaacgggtgtcccgtcattgccgtctgaagaggagtcctcagtctacgtggtac

SEQ ID NO: 97 - DNA, 94 bp, Homo sapiens
  source 1..94: mol_type = unassigned DNA
cctgcccagggcctcaccaccaacttcatccacgttcaccttgccccacagggcagtaacggcagacttctcctcaggagtcagatgcaccatg

SEQ ID NO: 98 - DNA, 23 bp, synthetic construct
  misc_feature 1..23: note = Figure 4 primer at position/strand 76/fw
  source 1..23: mol_type = other DNA
gtaacggcagacttctcctcagg

SEQ ID NO: 99 - DNA, 23 bp, synthetic construct
  misc_feature 1..23: note = Figure 4 primer at postion/strand 45/rev
  source 1..23: mol_type = other DNA
gtctgccgttactgccctgtggg

(19) United States
(12) Patent Application Publication
(10) Pub. No.: US 2025/0084437 A1
(43) Pub. Date: Mar. 13, 2025

(54) RECOMBINANT LENTIVIRAL VECTOR FOR STEM CELL-BASED GENE THERAPY OF SICKLE CELL DISORDER

(71) Applicants: INSTITUT NATIONAL DE LA SANTE ET DE LA RECHERCHE MEDICALE (INSERM), Paris (FR); UNIVERSITE PARIS DESCARTES, Paris (FR); ASSISTANCE PUBLIQUE-HOPITAUX DE PARIS, Paris (FR); IMAGINE - INSTITUT DES MALADIES GENETIQUES NECKER ENFANTS MALADES, Paris (FR)

(72) Inventors: Annarita MICCIO, Paris (FR); Vasco MENEGHINI, Bretigny-Sur-Orge (FR)

(21) Appl. No.: 18/906,538

(22) Filed: Oct. 4, 2024

Related U.S. Application Data
(62) Division of application No. 16/618,226, filed on Nov. 29, 2019, now Pat. No. 12,139,720.

(30) Foreign Application Priority Data
Jun. 2, 2017 (EP) 17305647.4

(51) Int. Cl. C12N 15/86; A61K 38/42; C12N 5/074; C12N 9/22

(52) U.S. Cl. CPC C12N 15/86; A61K 38/42; C12N 5/0607; C12N 9/22; C12N 2310/20; C12N 2740/15043; C12N 2830/40; C12N 2830/48

(57) ABSTRACT

This invention relates to recombinant lentiviral vectors, compositions thereof, the use of the vectors or the compositions thereof, kits of parts comprising said vectors or compositions thereof and a catalytically active Cas9 or Cpf1 protein, methods for modifying the genome of a hematopoietic stem/progenitor cell (HSPC), and the HSPC obtainable by such methods.

### CROSS-REFERENCE TO RELATED APPLICATIONS

This application is a divisional of U.S. application Ser. No. 16/618,226, filed Nov. 29, 2019, which is a U.S. National Stage Application of International Application No. PCT/EP2018/064531, filed on Jun. 1, 2018, which claims priority to European Patent Application No. 17305647.4, filed on Jun. 2, 2017, the entire disclosures of which are hereby incorporated by reference in their entireties.

### SEQUENCE LISTING

The instant application contains a Sequence Listing which has been submitted electronically in XML file format and is hereby incorporated by reference in its entirety. Said XML copy, created on Sep. 12, 2024, is named 0061_0011-DIV_SL.xml and is 206,988 bytes in size.

### BACKGROUND OF THE INVENTION

Sickle cell disease (SCD) is amongst the most common inherited blood disorders. The disease is caused by a single amino acid substitution of a polar glutamic acid with a nonpolar valine residue in the beta-globin chain. At low oxygen tensions, this residue interacts with a hydrophobic pocket (aa 85-88) on the surface of a second Hb tetramer, thus leading to the polymerization of sickle hemoglobin (HbS) and formation of sickle Red Blood Cells (RBC). This can cause occlusions of small blood vessels, leading to impaired oxygen delivery to tissues, pain crises, respiratory complications, and organ damage, often associated with a high mortality rate. SCD represents an increasing health problem in Mediterranean, American and Asian countries. In particular, SCD has become one of the most common genetic diseases in France (birth prevalence of 1:2,400).

Currently, the main treatment involves symptomatic care and transfusion of RBC as clinically necessary. However, the use of regular transfusion therapy can lead to significant side effects, such as iron overload, which can even occur when the strictest iron-chelation regimens are used.

The clinical course of SCD is also improved in the presence of elevated expression of the fetal gamma-globin genes. Indeed, in SCD, fetal gamma-globin can exert a potent anti-sickling effect decreasing the incidence of vaso-occlusions. Therefore, the expectation is that pharmacological treatments that increase gamma-globin expression would benefit SCD patients. However, pharmacological treatments are not equally effective for all patients and are associated with considerable toxicity. There are clinical data showing that high expression of delta globin results in mild disease phenotype in SCD patients

The only definitive cure for SCD is allogeneic hematopoietic stem/precursor cell (HSPC) transplantation, which is, however, available to a small proportion (<30%) of the patients with an HLA-compatible donor.

Therefore, new approaches remain highly desired because such treatments are not equally effective for all patients, are associated with considerable toxicity and do not represent a definitive treatment.

Autologous transplantation of genetically corrected HSPC is considered an attractive therapeutic option, for example it is an alternative for patients lacking a compatible donor. Transplantation of autologous genetically corrected HSPC is currently based on the use of lentiviral vectors expressing a mutated beta-globin gene with anti-sickling properties and showed a partial correction of the SCD phenotype in human RBC (Romero et al., J Clin Invest, 2013, 23(8):3317-30). Lentivirus vectors (LVs) expressing the human beta-globin gene have been successfully used in preclinical murine models and in human thalassemic cells (Imren et al., Proc Natl Acad Sci USA, 2002, 99(22):14380-5; Miccio et al., Proc Natl Acad Sci USA, 2008,105(30):10547-52; Miccio et al., PLoS One, 2011, 6(12):e27955; Puthenveetil et al., Blood, 2004,104(12):3445-53). Therapeutic approaches based on the use of gamma-globin expressing LVs have also been proposed, leading to the improvement of the murine SCD phenotype (Perumbeti et al., Blood, 2009, 114(6):1174-85). Early clinical data indicate that the expression of an anti-sickling beta-globin molecule harboring an aminoacid derived from the gamma-globin chain in erythrocytes provides some degree of clinical benefit in beta-thalassemic patients and in a SCD patient (Ribeil J A, abstract n.2311, 58^{th} annual meeting ASH, 2016) (Ribeil et al. N Engl J Med, 2017,376(9):848-55). However, to achieve the functional correction of a high number of SCD RBC, it is mandatory to find new gene therapy approaches. Indeed, despite the undeniable progress in the gene therapy field, the treatment of SCD requires further key improvements to achieve the functional correction of a high number of SCD RBC in order to cure SCD.

The applicant now proposes a new approach to achieve these therapeutic goals through a novel gene therapy/genome editing combined strategy based on a high-titer lentiviral vector carrying: (i) the anti-sickling globin transgene under the control of the endogenous beta-globin promoter and LCR enhancers; (ii) a gRNA targeting the endogenous HBB (beta-hemoglobin) gene or gRNA resulting in an inhibition of the repression of gamma globins. LV transduction of a large proportion of patient hematopoietic stem cells (HSC) followed by transient Cas9 delivery will permanently alter the endogenous sickle HBB gene, ensuring meanwhile the expression of globin transgene in genome-edited cells.

### SUMMARY OF THE INVENTION

The inventors propose here new recombinant lentiviral vectors and processes for autologous gene therapy that are particularly efficient and easy to practice for both incorporating a therapeutic DNA into a patient's cell and to knock out an altered gene in said patient's cells.

The invention relates to a recombinant lentiviral vector comprising in its genome:
(i) a nucleotide sequence encoding a protein that has a therapeutic effect, said protein being selected from the group consisting of gamma-globin, beta-globin, delta-globin and variants thereof; and
(ii) a nucleotide sequence encoding a guide RNA (gRNA) that comprises a spacer adapted to bind to a target nucleotide sequence, said target nucleotide sequence is
   a) within the coding sequence or within a transcribed non-coding sequence of a target gene, said target gene is selected from beta-globin gene and BCL11A gene, or
   b) within the promoter region of a target gene, said target gene is gamma-globin gene.

The invention also relates to a composition comprising a vector according to the invention or a plurality of vectors according to the invention.

The invention also relates to a kit of parts comprising:
a vector of the invention or a composition of the invention; and
a catalytically active Cas9 or Cpf1 protein or a nucleotide sequence encoding a catalytically active Cas9 or Cpf1 protein.

The invention also relates to the use of a recombinant lentiviral vector of the invention or a composition of the invention or a kit of the invention for introducing into a hematopoietic stem/progenitor cell (HSPC):
(i) the nucleotide sequence encoding a protein that has a therapeutic effect, said protein being selected from the group consisting of gamma-globin, beta-globin, delta-globin and variants thereof; and
(ii) the nucleotide sequence encoding a guide RNA (gRNA) that comprises a spacer adapted to bind to a target nucleotide sequence, said target nucleotide sequence is
   a. within the coding sequence or within a transcribed non-coding sequence of a target gene, said target gene is selected from beta-globin gene and BCL11A gene, or
   b. within the promoter region of a target gene, said target gene is gamma-globin gene.

The invention also relates to a method for modifying the genome of hematopoietic stem/progenitor cells (HSPC), in vitro or ex vivo, comprising the steps of:
a) contacting a HSPC with a recombinant lentiviral vector of the invention or a composition of the invention to obtain a transduced HSPC, wherein the lentiviral vector is integrated into the genome of said HSPC; and
b) introducing into the transduced HSPC a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced HSPC.

The invention also relates to a method for preparing a genetically modified hematopoietic stem/progenitor cell (HSPC), in vitro or ex vivo, comprising the steps of:
a) contacting a HSPC with a recombinant lentiviral vector of the invention or a composition of the invention to obtain a transduced HSPC, wherein the lentiviral vector is integrated into the genome of said HSPC; and
b) introducing into the transduced HSPC a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced HSPC.

The invention also relates to a genetically modified HSPC obtainable by the methods of the invention.

### DETAILED DESCRIPTION OF THE INVENTION

### Recombinant Vector

The invention relates to a recombinant lentiviral vector comprising in its genome:
(i) a nucleotide sequence encoding a protein that has a therapeutic effect, said protein being selected from the group consisting of gamma-globin, beta-globin, delta-globin and variants thereof; and
(ii) a nucleotide sequence encoding a guide RNA (gRNA) that comprises a spacer adapted to bind to a target nucleotide sequence, said target nucleotide sequence is:
   a. within the coding sequence or within a transcribed non-coding sequence of a target gene, said target gene is selected from beta-globin gene and BCL11A gene, or
   b. within the promoter region of a target gene, said target gene is gamma-globin gene.

In some embodiments, said target nucleotide sequence is within a coding sequence or within a non-coding sequence implied in the expression of BCL11A gene, such as erythroid-specific BCL11A intronic enhancer.

Lentiviruses are used as a vector or delivery system for the transfer of nucleotide sequences to a cell. The transfer can occur in vitro, ex vivo or in vivo. When used in this fashion, the lentiviruses are typically called “lentiviral vectors”.

The lentiviral vector according to the invention is a virus particle that contains a lentivirus-derived viral genome, lacks self-renewal ability, and has the ability to introduce a nucleotide sequence into a cell. The recombinant lentiviral vector of the invention is therefore a “recombinant lentiviral integrative vector”.

The lentiviral vector may be derived from complex retroviruses such as the human immunodeficiency virus (HIV). In the present invention, lentiviral vectors derived from any strain and subtype can be used. The lentiviral vector may be based on a human or primate lentivirus, such as HIV, or a non-human lentivirus such as feline immunodeficiency virus, simian immunodeficiency virus or equine infectious anemia virus (EIAV). In a preferred embodiment, the lentiviral vector is a HIV-based vector and especially a HIV-1-based vector.

“Recombinant” is used consistently with its usage in the art to refer to a nucleotide sequence that comprises portions that do not naturally occur together as part of a single sequence or that have been rearranged relative to a naturally occurring sequence. A recombinant nucleotide sequence (or transgene) is created by a process that involves the human intervention and/or is generated from a nucleic acid that was created by human intervention (e.g., by one or more cycles of replication, amplification, transcription, etc.). A recombinant virus is one that comprises a recombinant nucleotide sequence.

A recombinant cell is one that comprises in its genome a recombinant nucleotide sequence. Thus, a “recombinant lentiviral vector” according to the invention refers to a lentiviral vector comprising in its genome a recombinant nucleotide sequence (or transgene).

The recombinant lentiviral vector “genome”, as used herein, accordingly contains, apart from the so-called recombinant nucleotide sequences placed under control of proper regulatory sequences for its expression, the sequences of the original lentivirus genome which are non-coding regions of said genome, and are necessary to provide recognition signals for DNA or RNA synthesis and processing (mini-viral genome). These sequences are cis-acting sequences necessary for packaging, reverse transcription and transcription and furthermore they contain a functional sequence favoring nuclear import in cells and accordingly transgenes transfer efficiency in said cells, which element is described as a DNA Flap element.

The lentiviral vector can be based on any suitable lentivirus which is able to deliver genetic information to a hematopoietic stem/progenitor cell (HSPC), in particular a human HSPC.

In the lentiviral vector of the present invention, the recombinant nucleotide sequences encode (i) a protein that has a therapeutic effect, said protein being selected from the group consisting of gamma-globin, beta-globin, delta-globin and variants thereof, and (ii) a gRNA that comprises a spacer adapted to bind to a target nucleotide sequence, said target nucleotide sequence is:
a. within the coding sequence or within a transcribed non-coding sequence of a target gene, said target gene is selected from beta-globin gene and BCL11A gene, or
b. within the promoter region of a target gene, said target gene is gamma-globin gene.

The official symbols of beta-like globin genes are: HBB (beta-globin gene), HBD (delta-globin gene), HBG1 and HBG2 (gamma-globin genes), HBA1 and HBA2 (alpha-globin genes). The Greek symbols (e.g. α, β, γ and δ) and the corresponding denomination (e.g. alpha, beta, gamma, and delta) are used independently in the present description. Furthermore, the beta-like globin genes/mRNA/proteins are independently used in italic or not in the present description (e.g. HBB gene or HBB gene; HBB mRNA and HBB mRNA and HBB protein or HBB protein).

The term “gamma-globin target gene” means HBG1, HBG2 or both HBG1 and HGB2.

The terms “protein that has a therapeutic effect” means a protein that provides an effect which is judged to be desirable and beneficial to a patient, in particular a patient with a sickle cell disease (SCD). Examples of a protein that has a therapeutic effect in the present invention may be the functional protein of a protein that has become dysfunctional due to a sickle cell disease (SCD). Thus, in one embodiment, the term “protein that has a therapeutic effect” refers to a protein that does not induce a sickle cell disease (SCD), and which is effective to provide therapeutic benefits to a patient, in particular a patient with a sickle cell disease (SCD). The protein that has a therapeutic effect may be a wild-type (WT) protein appropriate for a patient with a sickle cell disease (SCD) to be treated, or it may be a mutant form of the WT protein (i.e. a variant of the WT protein) appropriate for a patient to be treated. In another embodiment the terms “protein that has a therapeutic effect” refer to a protein that may be incorporated within hemoglobin tetramers and does not allow the hemoglobin tetramer polymerization in which SCD originates.

For example, a globin (e.g. a beta-like globin) that has a therapeutic effect refers to a globin protein that does not produce a hemoglobinopathy phenotype, and which is effective to provide therapeutic benefits to a patient defective for said globin. A globin that has a therapeutic effect may be a wild-type globin appropriate for a patient to be treated, or variant thereof, preferably a variant which provides for superior properties, for example superior anti-sickling properties.

All the globin variants are encoded by a transgene containing the introns of the beta-globin gene, the gamma-globin gene and/or the delta-globin gene, preferably the intron of the beta-globin gene, as contained in the GLOBE vector (Miccio et al., Proc Natl Acad Sci USA, 2008,105(30):10547-52). In some embodiments, the second intron harbors a 600-bp RsaI to SspI deletion.

In some embodiments, the globin variant has one or more mutations that increase the anti-sickling properties of the protein, for example:
beta-globin comprising one mutation Thr87Gln (i.e. beta-globin wild-type sequence having threonine replaced by glutamine at position 87, named Beta AS1 (T87Q), encoded by SEQ ID NO: 1) (Ribeil J A, abstract n.2311, 58^{th} annual meeting ASH, 2016) (Ribeil et al. N Engl J Med, 2017,376(9):848-55),
beta-globin comprising three mutations Gly16Asp, Glu22Ala and Thr87Gln (i.e. beta-globin wild-type sequence having glycine replaced by aspartic acid at position 16; Glutamic acid replaced by alanine at position 22 and threonine replaced by glutamine at position 87, named Beta AS3, encoded by SEQ ID NO: 2) (Romero et al., J Clin Invest, 2013, 23(8):3317-30),
gamma-globin comprising two mutations Gly16Asp and Glu22Ala (gamma-globin wild-type sequence having glycine replaced by aspartic acid at position 16 and glutamic acid replaced by alanine at position 22, named Gamma-beta hybrid AS2 (G16D and D22A), encoded by SEQ ID NO: 4), or
delta-globin comprising one mutation Gly16Asp (i.e. delta-globin wild-type sequence having glycine replaced by aspartic acid at position 16, named Delta-beta hybrid AS1 (G16D), encoded by SEQ ID NO: 6).

In a specific embodiment, Gamma-beta hybrid AS2 (G16D and D22A) may be A-Gamma-beta hybrid AS2 (G16D and D22A), i.e. the protein encoded by the genes HBG1, or G-Gamma-beta hybrid AS2 (G16D and D22A), i.e. the protein encoded by the genes HGB2, preferably A-Gamma-beta hybrid AS2 (G16D and D22A).

In a preferred embodiment, the protein that has a therapeutic effect is gamma-globin, beta-globin or a variant thereof, for example a variant having anti-sickling properties. Preferably, the protein that has a therapeutic effect is beta-globin or a variant thereof.

In a specific embodiment, the intended patient is a mammalian being, preferably a human being, regardless of age and gender. In particular, the patient has a sickle cell disease (SCD). Thus, in a specific embodiment, the protein that has a therapeutic effect is selected from the group consisting of human beta-globin, human gamma-globin, human delta-globin and variants thereof, in particular human beta-globin or human gamma-globin, preferably human beta-globin. The variants thereof may be:
Beta AS1 (T87Q), encoded by SEQ ID NO: 1;
Beta AS3, encoded by SEQ ID NO: 2;
Gamma-beta hybrid, encoded by SEQ ID NO: 3;
Gamma-beta hybrid AS2 (G16D and D22A), encoded by SEQ ID NO: 4;
Delta-beta hybrid, encoded by SEQ ID NO: 5; or
Delta-beta hybrid AS1 (G16D), encoded by SEQ ID NO: 6.

In a specific embodiment, beta-globin, gamma-globin, delta-globin and their variants can harbor silent mutations that impair the gRNA binding to the transgene, for example:
Beta AS1 (T87Q) modified to avoid targeting by gRNA D, encoded by SEQ ID NO: 7; or
Beta AS3 modified to avoid targeting by gRNA D, encoded by SEQ ID NO: 8.

In some embodiments, the protein that has a therapeutic effect is involved in a sickle cell disease (SCD) when said protein is altered in a patient.

The terms “protein is altered” or “altered protein” means a change (increase or decrease) in the expression levels and/or activity of the protein and/or a structural change in the protein. An altered protein may cause a sickle cell disease (SCD). In particular, for SCD, mutation in beta-globin increases the propensity of hemoglobin tetramers to polymerize. Said “altered protein” is encoded by an “altered gene” (e.g. altered HBB gene).

According to the invention, the gRNA comprises a spacer (said spacer is also called “CRISPR spacer” or “gRNA spacer” in the present description) adapted to bind to a target nucleotide sequence. The terms “target nucleotide sequence” means any endogenous nucleic acid sequence of the genome of a cell, such as, for example a gene or a non-coding sequence within or adjacent to a gene, in which it is desirable to modify it by targeted non-homologous end-joining (NHEJ) or MMEJ (Microhomology-mediated end-joining), in particular to disrupt (e.g. to knock-out) the expression and/or the function of said gene (also called “target gene”). The target nucleotide sequence can be present in a chromosome. In some embodiments, the target nucleotide sequence is within the coding sequence of the target gene or within a transcribed non-coding sequence of the target gene such as, for example, leader sequences, trailer sequence or introns. According to the invention, the target gene is known to be involved in a sickle cell disease (SCD) when said target gene is expressed in a patient. In a specific embodiment, the target gene is betaS-globin gene. The term “betaS-globin gene” means the altered HBB gene in SCD patients. The “betaS-globin gene” comprises a mutation A>T in the seventh codon of the beta-globin gene (in the sixth codon of the beta globin in the old nomenclature).

Generally, the nucleotide sequence encoding the gRNA is designed to encode a gRNA that may disrupt the expression and/or the function of a target gene through the insertion of frameshift mutations in its coding sequence. Thus, the nucleotide sequence encoding the gRNA is designed to encode a gRNA that may disrupt the function and/or the expression of a target protein. This disruption takes place when said gRNA forms a complex with Cas9 or Cpf1 in the transduced cell through the CRISPR/Cas9 system or CRISPR/Cpf1 system respectively (see below).

The gRNA can also be designed to reproduce small deletion detected in Hereditary Persistent of Fetal Hemoglobin (HPFH) patients (e.g. 13 bp small deletion; Traxler E A et al., Nat Med, 2016, 22(9):987-90) within the promoters of HBG1 and HBG2. Accordingly, in a specific embodiment of the present invention, the recombinant lentiviral vector comprises in its genome a nucleotide sequence encoding a guide RNA (gRNA) that comprises a gRNA spacer adapted to generate a mimetic effect to the one of 13 bp small deletion within the promoters of HBG1 and HBG2. In some embodiments, said gRNA spacer is encoded by SEQ ID No: 73 (5′-CTTGTCAAGG CTATTGGTCA-3′).

In a specific embodiment of the present invention, the recombinant lentiviral vector comprises in its genome a nucleotide sequence encoding a guide RNA (gRNA) that comprises a gRNA spacer adapted to bind to the wild-type or the altered HBB gene. In some embodiments, the gRNA spacer is encoded by a nucleotide sequence selected from SEQ ID No: 23 to SEQ ID No: 36.

In another specific embodiment of the present invention, the recombinant lentiviral vector comprises in its genome a nucleotide sequence encoding a guide RNA (gRNA) that comprises a gRNA spacer adapted to bind to BCL11A gene. In a specific embodiment, said guide RNA (gRNA) comprises a gRNA spacer adapted to bind the intronic erythroid specific enhancer of BCL11A gene. In some embodiments, the gRNA spacer is encoded by the nucleotide sequence SEQ ID NO: 74) (5′-CACAGGCTCCAGGAAGGGTT-3′).

Thus, according to the invention, the recombinant lentiviral vector provides expression of the protein that has a therapeutic effect and of the gRNA into a hematopoietic stem/progenitor cell (HSPC) transduced by said vector (also called “transduced HSPC”). The transduced HSPC therefore expresses a gRNA that may disrupt the gene and, as a consequence, the function and/or expression of a target protein in the transduced HSPC by forming a complex with Cas9 or Cpf1.

The terms “disrupt the function of a target protein” or “target protein is disrupted” or “disrupted target protein” means a decrease in the expression levels and/or activity of the target protein.

The terms “disrupt the function of a target gene” or “target gene is disrupted” or “disrupted target gene” means a decrease in the expression level and/or function of the target gene.

The term “to disrupt” comprises “to knock out”. In a specific embodiment, the gRNA disrupts the expression and/or the function of the target gene and therefore the gRNA disrupts the expression and/or the activity of the target protein. In a specific embodiment, the target gene is a gene coding for the betaS-globin (i.e. betaS-globin gene).

In a preferred embodiment, the recombinant lentiviral vector of the invention further comprises the elements 1, 2, 3, 4 and 5 below, or elements 1, 2, 3, 4, 5, and 6 below:
1) An expression cassette encoding the protein that has a therapeutic effect;
2) A self-inactivating (SIN) LTR configuration;
3) A packaging signal;
4) A Rev Responsive Element (RRE) to enhance nuclear export of unspliced recombinant lentiviral vector RNA;
5) A central polypurine tract (cPPT) to enhance nuclear import of recombinant lentiviral vector genomes; and
6) A post-transcriptional regulatory element (PRE) to enhance recombinant lentiviral vector genome stability and to improve recombinant lentiviral vector titers (e.g. WPRE).
   An Expression Cassette Encoding the Protein that has a Therapeutic Effect

As indicated above, in various embodiments the recombinant lentiviral vector described herein comprises an expression cassette encoding the protein that has a therapeutic effect. For example, the expression cassette is a beta-like globin gene (i.e. gamma-globin gene, beta-globin gene or delta-globin gene, or variants thereof) cassette which encodes the protein that has a therapeutic effect. For example, the expression cassette encodes a human globin, for example the expression cassette comprises ˜1.95 kb recombinant human gamma-beta hybrid globin gene (i.e. gamma-globin exons and beta-globin introns, where beta-globin intron 2 has a 600-bp RsaI to SspI deletion) under the control of transcriptional control elements (e.g. the human beta-globin gene promoter (e.g., −265 bp/+50 bp)), and a 2.7 kb composite human beta-globin locus control region (e.g., HS2 −1203 bp; HS3 −1213 bp and/or HS4 −954 bp).

The beta-like globin gene (i.e. beta-globin gene, gamma-globin gene, delta-globin gene, or variants thereof) cassette, however, is illustrative and need not be limiting. Using the known cassette described herein, numerous variations will be available to one of skill in the art. Such variations include, for example, further and/or alternative mutations to the beta-globin to further enhance non-sickling properties (e.g., PAS3 cassette is described by Levasseur (2003) Blood 102: 4312-4319), alterations in the transcriptional control elements (e.g., promoter and/or enhancer such as HS4), variations on the intron size/structure, and the like. In a preferred embodiment, the cassette lacks HS4 (i.e. the recombinant lentiviral vector lacks HS4). The inventors showed that the absence of HS4 increases recombinant lentiviral vector titer and therefore efficiency and efficacy of the recombinant lentiviral vector; and the absence of HS4 does not affect the therapeutic potential of the globin-expressing recombinant lentiviral vectors.

### Self Inactivating (SIN) LTR Configuration.

To further improve safety, in various embodiments, the recombinant lentiviral vectors described herein comprise a TAT-independent, self-inactivating (SIN) configuration. Thus, in various embodiments it is desirable to employ in the LVs described herein an LTR region that has reduced promoter activity relative to wild-type LTR. Constructs can be provided that are effectively “self-inactivating” (SIN), which provides a biosafety feature. SIN vectors are ones in which the production of full-length vector RNA in transduced cells is greatly reduced or abolished altogether. This feature minimizes the risk that replication-competent recombinants (RCRs) will emerge. Furthermore, it reduces the risk that cellular coding sequences located adjacent to the recombinant lentiviral vector integration site will be aberrantly expressed. The SIN configurations are well known in the art.

### Packaging Signal.

In various embodiments the recombinant lentiviral vectors described herein further comprise a packaging signal. A “packaging signal”, “packaging sequence”, or “psi sequence” is any nucleic acid sequence sufficient to direct packaging of a nucleic acid whose sequence comprises the packaging signal into a retroviral particle. The term includes naturally occurring packaging sequences and also engineered variants thereof. Packaging signals of a number of different retroviruses, including lentiviruses, are known in the art. In a specific embodiment, the packaging sequence is the naturally occurring packaging sequences.

### Rev Responsive Element (RRE).

In certain embodiments, the recombinant lentiviral vectors described herein comprise a Rev Response Element (RRE) to enhance nuclear export of unspliced RNA. RREs are well known to those of skill in the art.

### Expression-Stimulating Posttranscriptional Regulatory Element (PRE)

In certain embodiments, the recombinant lentiviral vectors described herein may comprise any of a variety of posttranscriptional regulatory elements (PREs) whose presence within a transcript increases expression of the heterologous nucleic acid (e.g., Gamma-beta hybrid globin gene) at the protein level. PREs may be particularly useful in certain embodiments, especially those that involve lentiviral constructs with poorly efficient promoters.

One type of PRE is an intron positioned within the expression cassette, which can stimulate gene expression. However, introns can be spliced out during the life cycle events of a lentivirus. Hence, if introns are used as PREs they are typically placed in an opposite orientation to the recombinant lentiviral vector genomic transcript. PREs are well known to those of skill in the art.

The recombinant lentiviral vector of the invention is able to provide expression of the gRNA into a hematopoietic stem/progenitor cell (HSPC) transduced by said recombinant lentiviral vector and is able to provide expression of the protein that has a therapeutic effect into an erythroblast derived from the transduced HSPC and/or into a differentiate progeny of the transduced HSPC.

In a specific embodiment, recombinant lentiviral vector is SEQ ID NO: 47, SEQ ID NO: 75 (LV.GLOBE-AS3modified.gRNA-BCL11Aenhancer), SEQ ID NO: 76 (LV.GLOBE-AS3modified.gRNA-13 bp-del) or SEQ ID NO: 94 (LV.GLOBE-AS3modified.gRNAD).

The invention also relates to a composition comprising a recombinant lentiviral vector of the invention or a plurality of recombinant lentiviral vectors of the invention. The recombinant lentiviral vector or a plurality of recombinant lentiviral vectors of the invention can be purified to become substantially pure. The terms “substantially pure” means that the recombinant lentiviral vectors contain substantially no replicable virus other than the recombinant lentiviral vectors. The purification can be achieved using known purification and separation methods such as filtration, centrifugation and column purification. If necessary, the recombinant lentiviral vector or a plurality of recombinant lentiviral vectors of the invention can be prepared as compositions by appropriately combining them with desired pharmaceutically acceptable carriers or vehicles. The term “pharmaceutically acceptable carrier” refers to a material that can be added to the recombinant lentiviral vector or the plurality of recombinant lentiviral vectors of the invention and does not significantly inhibit recombinant lentiviral vector-mediated gene transfer. Specifically, the recombinant lentiviral vector or the plurality of recombinant lentiviral vectors can be appropriately combined with, for example, sterilized water, physiological saline, culture medium, serum, and phosphate buffered saline (PBS). The recombinant lentiviral vector or the plurality of recombinant lentiviral vectors can also be combined with a stabilizer, biocide, etc. Compositions containing a recombinant lentiviral vector or a plurality of recombinant lentiviral vectors of the present invention are useful as reagents or pharmaceuticals. For example, compositions of the present invention can be used as reagents for gene transfer into a cell, preferably for transduction of a hematopoietic stem/progenitor cell (HSPC), in particular a human HSPC.

The invention also relates to a kit of parts comprising:
a recombinant lentiviral vector of the invention or a composition of the invention; and
a catalytically active Cas9 or Cpf1 protein or a nucleotide sequence encoding a catalytically active Cas9 or Cpf1 protein.

According to the invention, the terms “catalytically active Cas9 or Cpf1” means either a “wild-type version of Cas9 or Cpf1” or a “catalytically active variant of Cas9 or Cpf1”.

According to the invention, a complex gRNA/Cas9 or gRNA/Cpf1 induces the target nucleotide sequence to be removed and/or new ones added through a system called “CRISPR/Cas9 system” or “CRISPR/Cpf1 system”. CRISPR means Clustered Regularly Interspaced Short Palindromic Repeats.

The CRISPR/Cas system is a prokaryotic immune system that confers resistance to foreign genetic elements such as those present within plasmids and phages, and provides a form of acquired immunity. CRISPR associated proteins (Cas), e.g. Cas9, use the CRISPR spacers to recognize and cut a target nucleotide sequence. By delivering into a cell the Cas9 and gRNA that comprises a spacer adapted to bind to a target nucleotide sequence, the cell genome can be cut at a desired location, inducing a target nucleotide sequence to be removed and/or new ones added (Mandal et al., Cell Stem Cell, 2014, 15(5):643-52).

According to the invention, said target nucleotide sequence is within the coding sequence of the target gene, within a transcribed non-coding sequence of the target gene. Therefore, the complex gRNA/Cas9 or gRNA/Cpf1 may disrupt (e.g. may induce knock-out of) the target gene.

It is well disclosed in the art that CRISPR/Cas9 system, when utilized for genome editing, may include Cas9, CRISPR RNA (crRNA) and trans-activating crRNA (tracrRNA).
crRNA comprises the RNA that binds to a target nucleotide sequence, said RNA is along with a tracrRNA (generally in a hairpin loop form);
tracrRNA and crRNA form an active complex, named guide RNA (gRNA). Because eukaryotic systems lack some of the proteins required to process crRNA, the synthetic construct gRNA was created to combine the essential pieces of RNA for Cas9 targeting into a single RNA. Commonly, the gRNA is expressed with the RNA polymerase type III promoter U6 (promoter U6);
Cas9 is a nuclease protein whose active form is able to modify DNA. Many variants exist with differing functions (i.e. single strand nicking, double strand break, DNA binding) due to Cas9's DNA site recognition function. In a preferred embodiment of the invention, Cas9 has a double strand break function.

The nucleic acid cleavages caused by Cas9 or Cpf1 are commonly repaired through the distinct mechanisms of homologous recombination or non-homologous end joining (NHEJ). NHEJ is an imperfect repair process that often results in changes to the nucleotide sequence at the site of the cleavage (i.e. the target nucleotide sequence). Repair via non-homologous end joining (NHEJ) often results in small insertions or deletions and can be used for the creation of specific gene knock-outs.

According to the present invention, CRISPR/Cas9 or CRISPR/Cpf1 system modifies the genome of a hematopoietic stem/progenitor cell (HSPC), preferably a human HSPC. Thus, in one aspect of the present invention, CRISPR/Cas9 or CRISPR/Cpf1 system aims to induce knock-out of the target nucleotide sequence in the transduced HSPC, and therefore to disrupt (e.g. to induce knock-out of) the target gene in the transduced HSPC, and therefore to disrupt (e.g. to suppress) the expression and/or the activity of the target protein in transduced HSPC and/or in the differentiated progeny of the transduced HSPC, such as the erythroid progeny of the transduced HSPC.

The invention also relates to the use of a lentiviral recombinant vector of the invention or a composition of the invention for introducing into a hematopoietic stem/progenitor cell (HSPC) (i) the nucleotide sequence encoding a protein that has a therapeutic effect, said protein being selected from the group consisting of beta-globin, gamma-globin, delta-globin and variants thereof, and (ii) the nucleotide sequence encoding a guide RNA (gRNA) that comprises a spacer adapted to bind to a target nucleotide sequence, said target nucleotide sequence is within the coding sequence of a target gene or within a transcribed non-coding sequence of a target gene, said target gene being selected from the group consisting of beta-globin gene and gamma-globin gene.

In some embodiment, the use according to the invention is in vitro or ex vivo. In another embodiment, the use according to the invention is in vivo.

The invention also relates to a method for modifying the genome of a hematopoietic stem/progenitor cell (HSPC), in vitro or ex vivo, comprising the steps of:
a) contacting a HSPC with a recombinant lentiviral vector of the invention or a composition of the invention to obtain a transduced HSPC, wherein the lentiviral vector is integrated into the genome of said HSPC; and
b) introducing into the transduced HSPC a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced HSPC.

The invention also relates to a method for preparing a genetically modified hematopoietic stem/progenitor cell (HSPC), in vitro or ex vivo, comprising the steps of:
a) contacting a HSPC with a recombinant lentiviral vector of the invention or a composition of the invention to obtain a transduced HSPC, wherein the lentiviral vector is integrated into the genome of said HSPC; and
b) introducing into the transduced HSPC a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced HSPC.

The term “transduction”, according to the invention, means the process by which a foreign nucleotide sequence is introduced into the genome of a cell by a recombinant viral vector. According to the invention, a hematopoietic stem/progenitor cell (HSPC) transduced by the recombinant lentiviral vector of the invention, also referred as a “transduced HSPC”, encodes (i.e. comprises in its genome) the nucleotide sequence encoding the protein that has a therapeutic effect and the nucleotide sequence encoding a gRNA that comprises a spacer adapted to bind to the target nucleotide sequence. Thus, according to a preferred embodiment, a transduced HSPC expresses the protein that has a therapeutic effect and the gRNA that comprises a spacer adapted to bind to the target nucleotide sequence.

The methods of the invention involve introducing a catalytically active Cas9 or Cpf1 protein (hereafter “Cas9” or “Cpf1”) or a nucleotide sequence encoding Cas9 or Cpf1, preferably a RNA encoding Cas9 or Cpf1, into the transduced HSPC. The following paragraphs only refer to “Cas9”, however, “Cas9” can be replaced by “Cfp1”.

According to the invention, Cas9 can be optimized for the organism in which it is being introduced. Thus, for example, Cas9 polynucleotide sequence derived from the S. pyogenes (Cas9 recognizing the NGG Protospacer adjacent motif (PAM), mutant VQR Cas9 recognizing the NGA PAM, mutant VRER recognizing the NGCG PAM), S. Thermophilus, N. Meningitidis or S. Aureus codon optimized for use in human cells is set forth in Cong et al., Science, 2013,339(6121):819-23; Mali et al., Science, 2013,339(61210):823-6.; Kleinstiver et al., Nature, 2015,523(7561):481-5; Hou et al., Proc Natl Acad Sci USA, 2013,110(39):11644-9; Ran et al., Nature, 2015,520(7546):186-191.

Cas9 may be directly introduced into the transduced HSPC as a protein or may be synthesized (or expressed) in situ in the cell as a result of the introduction of a nucleotide sequence encoding Cas9, for example a DNA or a RNA encoding Cas9, preferably a RNA encoding Cas9.

Cas9 or a nucleotide sequence encoding Cas9 can be produced outside the cell and then introduced thereto.

Methods for introducing a nucleotide sequence into cells are known in the art and including, as non-limiting examples, stable transduction methods wherein the nucleotide sequence is integrated into the genome of the cell (recombinant viral vector-mediated methods) or transient transfection methods wherein nucleotide sequence is not integrated into the genome of the cell (recombinant non-integrating viral vector-mediated methods, liposomes, microinjection, electroporation, particle bombardment and the like). Said nucleotide sequence may be included in a vector, more particularly a plasmid or a viral vector, in view of being expressed in the cells. In a preferred embodiment, the method for introducing a nucleotide sequence encoding Cas9 into HSPC is a transient transfection method.

In a specific embodiment, the nucleotide sequence encoding Cas9 is a DNA encoding Cas9. In this embodiment, the transient transfection is particularly advantageous because the DNA sequence encoding Cas9 is not integrated into the genome of the HSPC and therefore Cas9 is thus produced transiently in a limited period of time. After the transient production, given that the HSPC does not comprise in its genome a nucleotide sequence encoding Cas9, the cell does not produce Cas9 anymore. This is particularly advantageous when the HSPC is then used as a medicament. Furthermore, the rapid gRNA degradation in absence of Cas9 nuclease will avoid interferon response and apoptosis, improving therefore safety issues.

In another specific embodiment, the nucleotide sequence encoding Cas9 is a RNA encoding Cas9. The RNA also has the advantage of not being integrated into the genome of the HSPC. For example, a RNA encoding Cas9 is introduced by electroporation.

Methods for introducing a protein into cells are known in the art and include as non-limiting examples the use of liposomes, microinjection, electroporation or particle bombardment. For example, Cas9 may be introduced into the HSPC by electroporation.

In a particular embodiment, Cas9 is introduced into the cell as a protein. In this embodiment, Cas9 has the advantage of not being integrated into the genome of the cell and to be rapidly degraded. Cas9 expression can therefore be easily controlled. Furthermore, though reduced as exemplified in the experimental section, the potential risk for off-target activity is even more reduced because of this transient expression of Cas9 endonuclease activity. For example, Cas9 is introduced by electroporation or nanoparticles.

According to the invention, Cas9 may form a complex with the gRNA in the transduced HSPC. Said Cas9/gRNA complex may bind to the target nucleotide sequence and may therefore disrupt the expression or the function of the target gene. In a preferred embodiment, the Cas9/gRNA complex induces a knock-out of the expression or the function of the target gene. In a specific embodiment, the methods of the invention are particularly advantageous because, the only cells that are able to survive after the disruption of the target gene are those that comprise in their genome the nucleotide sequence encoding the protein that has a therapeutic effect and that express said protein that has a therapeutic effect. In this specific embodiment, the protein that has a therapeutic effect is needed by the cell to survive after the disruption of the target gene.

The invention also relates to a genetically modified HSPC obtainable by the methods according the invention and said genetically modified HSPC for use as a medicament.

In another embodiment, the invention relates to a genetically modified HSPC obtainable by the methods according the invention for use in the treatment of sickle cell disease (SCD).

The recombinant lentiviral vectors are particularly useful for the transduction of HSPC, obtained either from the bone marrow, the peripheral blood or the umbilical cord blood. Particularly preferred cells are CD34+ cells.

The invention also relates to a method of treating sickle cell disease (SCD) in a patient comprising the steps of:
a) obtaining a hematopoietic stem/progenitor cell (HSPC) from the patient;
b) contacting the HSPC with a recombinant lentiviral vector of any of claims 1 to 4 or a composition of claim 5 to obtain a transduced HSPC;
c) introducing into the transduced HSPC a catalytically active Cas9 or Cpf1 protein or a nucleotide sequence encoding a catalytically active Cas9 or Cpf1 protein, said catalytically active Cas9 or Cpf1 protein disrupts the expression and/or the function of the target gene when introduced or expressed into the transduced HSPC, to obtain a genetically modified HSPC; and
d) administrating the genetically modified HSPC to the patient

In one embodiment, a human HSPC can be removed from a human, e.g. a human patient, using methods well known to those of skill in the art and modified as noted above. The modified HSPC (i.e. the genetically modified HSPC) is then reintroduced into the same human.

In some embodiments, the administration may be a transplantation or an inoculation, in particular a transplantation or an inoculation in the bone narrow.

According to the invention, when the protein that has a therapeutic effect is a functional version (e.g. the wild-type version) of the target protein, the design of the nucleotide(s) sequence(s) (e.g. the nucleotide sequence encoding the protein that has a therapeutic effect and/or the nucleotide sequence encoding the gRNA) will be easily adapted by the skilled person in order to avoid that the gRNA targets the nucleotide sequence encoding the protein that has a therapeutic effect (codon design). For example, the recombinant lentiviral vector comprises a nucleotide sequence encoding beta-globin (e.g. PAS3 beta-globin cassette, described by Levasseur et al., Blood, 2003,102(13):4312-9 and a nucleotide sequence encoding a gRNA targeting the sickle beta-globin. In this case, to avoid the unwanted disruption of the nucleotide sequence encoding beta-globin (i.e. the beta-globin transgene), the nucleotide sequence encoding beta-globin will be modified introducing silent mutations in the transgene sequence, so that it will not be recognized by the gRNA (see for example SEQ ID NO: 7 and SEQ ID NO: 8, FIG. 14). To this aim, the skilled person commonly uses synonymous codons (coding for the same amino acids), allowing the change of the nucleotide sequence and the production of an identical beta-globin protein. Generally, synonymous codons will be chosen amongst the most frequently used codons in the beta- and alpha-globin genes.

### BRIEF DESCRIPTION OF THE DRAWINGS

FIG. 1: Construction of a recombinant lentiviral vector encoding a beta-like globin gene

FIG. 2: Evaluation of genome editing efficiency in hematopoietic cells using the CRISPR-Cas9 system

FIG. 3: Construction and screening of a gRNA for beta-globin gene inactivation: design of gRNAs targeting HBB gene. The amino acid sequence is set forth as SEQ ID NO: 95. The primer sequences are set forth as SEQ ID NOS: 49-60 (oriented in the 5′ to 3′ direction).

FIG. 4: Selection of gRNAs targeting the beta-globin gene: design of novel gRNAs The sequences depicted in step A are set forth as SEQ ID NO: 95 (amino acid) and SEQ ID NO: 96 (nucleotide). The sequence depicted in step C is set forth as SEQ ID NO: 97. The primer sequences depicted in step D are set forth as SEQ ID NOS: 98 and 99.

FIG. 5: Cleavage efficiency of gRNAs A, B, D and E in K562 and HUDEP-2 erythroid cell lines

FIG. 6: Down regulation of beta-globin expression in HUDEP-2

FIG. 7: Cleavage efficiency of selected gRNA (B, D and E) in HSPCs

FIG. 8: Down regulation of beta-globin expression in HSPC-derived erythroid cells

FIG. 9: Optimization of gRNA-mediated disruption of the target site

FIG. 10: Construction of a recombinant lentiviral vector according to the invention

FIG. 11: Transduction of HSPC with a recombinant lentiviral vector according to the invention and introduction of Cas9 into the transduced cell.

FIG. 12: Genetic modification of patient SCD HSPC in vitro

FIG. 13: Genetic modification of patient SCD HSC in vivo

FIG. 14: nucleotide sequences encoding globin variants that have a therapeutic effect according to the invention. The gRNA D target site is underlined. The nucleotides changes in the Beta AS3 (modified to avoid targeting by gRNA D) and Beta AS1 (T87Q) (modified to avoid targeting by gRNA D) transgenes are highlighted in grey/green.

FIG. 15: Assessment of globin mRNAs expression in mature erythroblasts (day 9 of differentiation) derived from control and genetically modified HUDEP-2 cell lines. UT: mature erythroblasts derived from non-transduced and non-transfected HUDEP-2 cells: “normal” level of globin β, δ and γ globin (negative control); VCN: «vector copy number»; Not transfected: mature erythroblasts derived from non-transfected HUDEP-2 cells; GFP+ (Cas9 plasmid): mature erythroblasts derived from HUDEP-2 cells expressing Cas9-GFP fusion protein, selected by FACS upon transfection with GFP-Cas9 plasmid; Cas9 protein: mature erythroblasts derived from HUDEP-2 cells transfected with Cas9-GFP protein without using selection-based strategies; when transduced, cells were treated with a lentiviral vector expressing beta-globin AS3mod transgene and a gRNA selected from: “D” lentiviral vector encoding optimized gRNA D, “luc” lentiviral vector encoding an optimized gRNA targeting the luciferase gene, which is not present in the human genome (negative control), “BCL11A” lentiviral vector encoding an optimized gRNA targeting the intronic erythroid-specific enhancer of BCL11A gene, “13bpdel” lentiviral vector encoding a gRNA designed to reproduce the 13 bp small HPFH deletion within the promoters of HBG1 and HBG2 genes; β: endogenous beta-globin mRNA; β-AS3: AS3 beta-globin transgene mRNA; Aγ+Gγ: gamma-globin mRNA; δ: delta-globin mRNA.

FIG. 16: Reverse phase HPLC profile of single globin chains in mature erythroblasts (day 9 of differentiation) derived from control and genetically modified HUDEP-2 cell lines. (A) mature erythroblasts derived from WT (wild-type) HUDEP-2 UT cells: not transduced and not transfected cells expressing “normal” level of globin β, δ and γ globin (negative control); (B) mature erythroblasts derived from HUDEP-2 cells transduced with LV.GLOBE.AS3mod-beta-globin.gRNA D (lentiviral GLOBE vector encoding the AS3modified beta-globin and the optimized gRNA D) but not transfected with Cas9-GFP plasmid: cells express the AS3modified beta-globin transgene and the endogenous beta-globin chain (no modification of the endogenous HBB gene); (C) mature erythroblasts derived from HUDEP-2 cells transduced cells with the LV.GLOBE.AS3mod-beta-globin.gRNA D (lentiviral GLOBE vector encoding the AS3modified beta-globin and the optimized gRNA D) and transfected with the GFP-Cas9 plasmid: cells express the AS3modified beta-globin transgene but not endogenous beta-globin chain because of the high rate of genome editing in the exon 1 of the endogenous HBB gene.

FIG. 17: Assessment of BCL11A mRNA expression (time-point analyses during differentiation) in HUDEP-2 cells transduced with a lentiviral vector encoding beta-globin AS3mod and a gRNA targeting the intronic erythroid-specific enhancer of BCL11A gene with (“+”) or without (“−”) transfection with Cas9-GFP plasmid.

FIG. 18: Reverse phase HPLC analysis of single globin chains in mature erythroblasts (day 9 of differentiation) derived from control and genetically modified HUDEP-2 cell lines. UT: mature erythroblasts derived from non-transduced and non-transfected HUDEP-2 cells: “normal” level of globin β, δ and γ globin (negative control); VCN: «vector copy number»; Not transfected: mature erythroblasts derived from non-transfected HUDEP-2 cells; GFP+(Cas9 plasmid): mature erythroblasts derived from HUDEP-2 cells expressing Cas9-GFP fusion protein, selected by FACS upon transfection with GFP-Cas9 plasmid; Cas9 protein: mature erythroblasts derived from HUDEP-2 cells transfected with Cas9-GFP protein without using selection-based strategies; when transduced, cells were treated with a lentiviral vector expressing AS3mod beta-globin transgene and a gRNA selected from: “D” lentiviral vector encoding optimized gRNA D, “luc” lentiviral vector encoding an optimized gRNA targeting the luciferase gene (negative control), “BCL11A” lentiviral vector encoding an optimized gRNA targeting the intronic erythroid-specific enhancer of BCL11A gene, “13bpdel” lentiviral vector encoding a gRNA designed to reproduce the 13 bp small HPFH deletion within the promoters of HBG1 and HBG2 genes; β: endogenous beta-globin chain; β-AS3: AS3 beta-globin chain; Aγ+Gγ: gamma-globin chains; δ: delta-globin chain

FIG. 19: Cation-exchange HPLC profile of hemoglobin tetramers in mature erythroblasts (day 9 of differentiation) derived from control and genetically modified HUDEP-2 cell line (A) WT (wild-type) HUDEP-2 UT cells: mature erythroblasts derived from non-transduced and non-transfected HUDEP-2 cells: “normal” level of globin HbA (hemoglobin tetramer containing the endogenous beta-globin chain), HbA2 (hemoglobin tetramer containing the endogenous delta-globin chain) and HbF (hemoglobin tetramer containing the endogenous gamma-globin chain) (negative control); mature erythroblasts derived from HUDEP-2 cells transduced with LV.GLOBE.AS3mod-beta-globin.gRNA D (lentiviral GLOBE vector encoding the AS3modified beta-globin and the optimized gRNA D) but not transfected with Cas9-GFP plasmid: cells express the Hb tetramer containing the AS3modified beta-globin transgene (HbAS3) and HbA containing the endogenous beta-globin chain (no modification of the endogenous HBB gene); (C) mature erythroblasts derived from HUDEP-2 cells transduced with the LV.GLOBE.AS3mod-beta-globin.gRNA D (lentiviral GLOBE vector encoding the AS3modified beta-globin and the optimized gRNA D) and transfected with the GFP-Cas9 plasmid: cells express HbAS3 but not HbA because of the high rate of genome editing in the exon 1 of the endogenous HBB gene.; (D) mature erythroblasts derived from HUDEP-2 cells transduced cells with the LV.GLOBE.AS3mod-beta-globin.gRNA 13 bp-del (lentiviral GLOBE vector encoding the AS3modified beta-globin and the optimized gRNA “13bpdel” encoding a gRNA designed to reproduce the 13 bp small HPFH deletion within the promoters of HBG1 and HBG2 genes) and transfected with the GFP-Cas9 plasmid: cells express the HbAS3, HbA and high levels of HbF upon genome editing of the promoters of HBG1 and HBG2 genes. HbA: α₂β₂ tetramers; HbAS3: α₂β-AS3₂ tetramers; HbA2: α₂δ₂ tetramers; HbF: α₂γ₂ tetramers.

FIG. 20: Quantification of hemoglobin tetramers by HPLC, as in FIG. 19, in mature erythroblasts (day 9 of differentiation) from control and genetically modified HUDEP-2 cell line. UT: mature erythroblasts derived from non-transduced and non-transfected HUDEP-2 cells: “normal” level of globin HbA, HbA2 and HbF (negative control); VCN: «vector copy number»; Not transfected: mature erythroblasts derived from HUDEP-2 cells non-transfected with GFP-Cas9 plasmid or Cas9-GFP protein; GFP+(Cas9 plasmid): mature erythroblasts derived from HUDEP-2 cells expressing Cas9-GFP fusion protein, selected by FACS upon transfection with GFP-Cas9; Cas9 protein: mature erythroblasts derived from HUDEP-2 cells transfected with Cas9-GFP protein without using selection-based strategies; when transduced, cells were treated with a lentiviral vector expressing beta-globin AS3mod transgene and a gRNA selected from: “D” lentiviral vector encoding optimized gRNA D, “luc” lentiviral vector encoding an optimized gRNA targeting the luciferase gene (negative control), “BCL11A” lentiviral vector encoding an optimized gRNA targeting the intronic erythroid-specific enhancer of BCL11A gene, “13bpdel” lentiviral vector encoding a gRNA designed to reproduce the 13 bp small HPFH deletion within the promoters of HBG1 and HBG2 genes. HbA: α₂β₂ tetramers; HbAS3: α₂β-AS3₂ tetramers; HbA2: α₂δ₂ tetramers; HbF: α₂γ₂ tetramers.

FIG. 21: HbF expression in mature erythroblasts (flow cytometry analysis on GPA(glycophorinA)^{high} populations) derived from control and genetically modified HUDEP-2 cells (day 9 of differentiation)

### EXAMPLES

### Example 1: Construction of a Recombinant Lentiviral Vector Encoding a Beta-Like Globin Gene

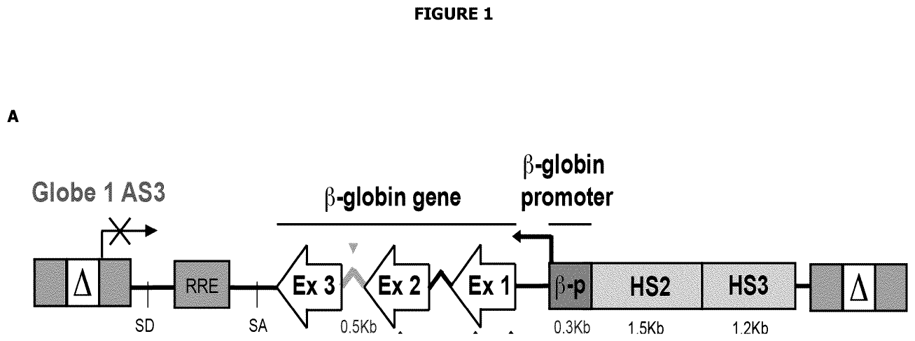

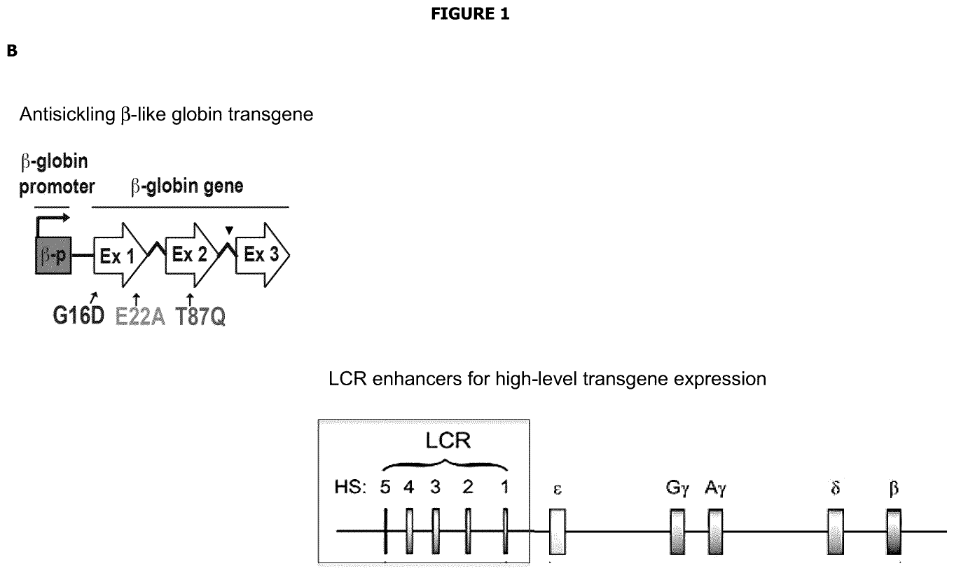

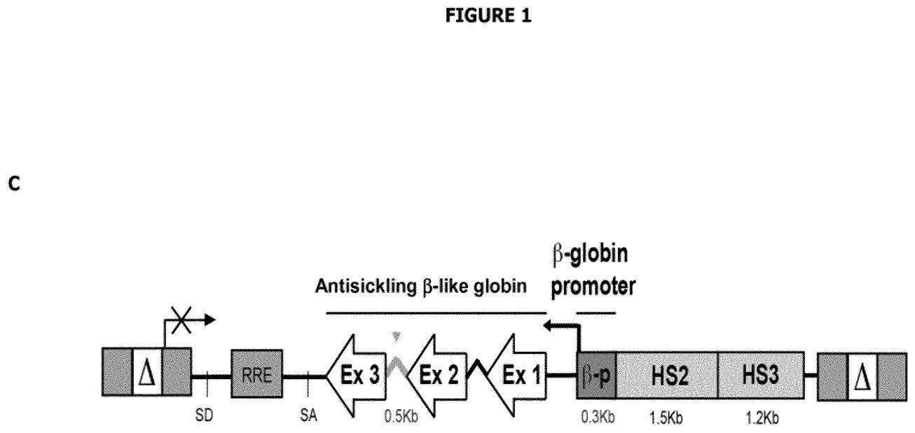

A recombinant lentiviral vector able to express at high levels a beta-like globin gene has been produced using the GLOBE lentiviral vector (Miccio et al., Proc Natl Acad Sci USA, 2008,105(30):10547-52, Roselli et al., EMBO Mol Med, 2010, 2(8):315-28). The GLOBE lentiviral vector in its proviral form contains LTRs deleted of 400 bp in the HIV U3 region (Δ), rev-responsive element (RRE), splicing donor (SD) and splicing acceptor (SA) sites, human beta-globin gene (exons and introns), beta-globin promoter (βp), and DNase I-hypersensitive sites HS2 and HS3 from beta-globin LCR (FIG. 1A and B). The construction of the recombinant lentiviral vector is detailed in FIG. 1C. An anti-sickling transgene (e.g. Beta AS3 (not modified), SEQ ID NO: 2; FIG. 1B) is included in the GLOBE lentiviral vector (FIG. 1C). The exons of the human beta-globin gene are replaced by exons of different anti-sickling transgenes (e.g. selected from SEQ ID NO: 1 to 8) by site-directed mutagenesis.

### Example 2: Evaluation of Genome Editing Efficiency in Hematopoietic Cells Using the CRISPR-Cas9 System

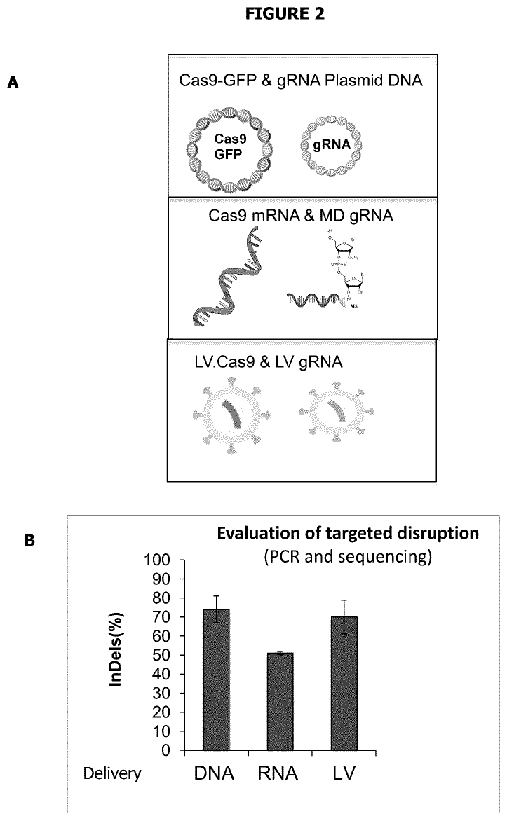

One million K562 hematopoietic cells were transfected with:
(i) 4 μg of a Cas9-GFP expressing plasmid (pMJ920, Addgene plasmid #42234) and 0.8 μg of a unrelated gRNA-expressing plasmid (MLM3636, Addgene plasmid #43860),
(ii) 20 μg of Cas9 mRNA modified with pseudouridine and 5-methylcytidine to reduce immune stimulation (Trilink, #L-6125) and 15 μg of chemically modified gRNAs (MD gRNA, 2′ O-Methyl unrelated gRNA, resistant to general base hydrolysis, Trilink); or
(iii) lentiviral vectors expressing Cas9 (Addgene, #52962) and an unrelated gRNA under the control of the human U6 promoter (FIG. 2A).

The above mentioned gRNAs were unrelated gRNAs, i.e. gRNAs binding regions which are not related to beta-globin gene or gamma-globin gene. In fact, the gRNA targets the gamma-delta intergenic region in the beta-globin locus (e.g. SEQ ID NO: 48).

K562 cells were transfected in a 100 μl volume using Nucleofector I (Lonza), the AMAXA Cell Line Nucleofector Kit V (Lonza, VCA-1003) and the T16 program.

After transfection, K562 cells were maintained in RPMI 1640 medium (Lonza) containing 2 mM glutamine and supplemented with 10% fetal bovine serum (FBS, BioWhittaker, Lonza), HEPES (20 mM, LifeTechnologies), sodium pyruvate (1 mM, LifeTechnologies) and penicillin and streptomycin (100 U/ml each, LifeTechnologies).

One week after transfection, DNA was extracted using PURE LINK Genomic DNA Mini kit (LifeTechnologies) following manufacturer's instructions. The genomic region encompassing the gRNA target site was amplified by PCR and subjected to Sanger sequencing. The genome editing efficiency (% InDels, frequency of small insertions and deletions), evaluated using TIDE (Tracking of In/Dels by Decomposition; (Brinkman et al., Nucleic Acids Res, 2014, 42(22):e168)) was higher than 50% for all the delivery systems (FIG. 2B).

These results showed that the use of DNA, RNA and lentiviral (LV) delivery systems for gRNA and Cas9 leads to a good editing efficiency in K562 hematopoietic cells.

### Example 3: Construction and Screening of a gRNA for Beta-Globin Gene Inactivation

1. Selection of gRNAs Targeting the Beta-Globin Gene

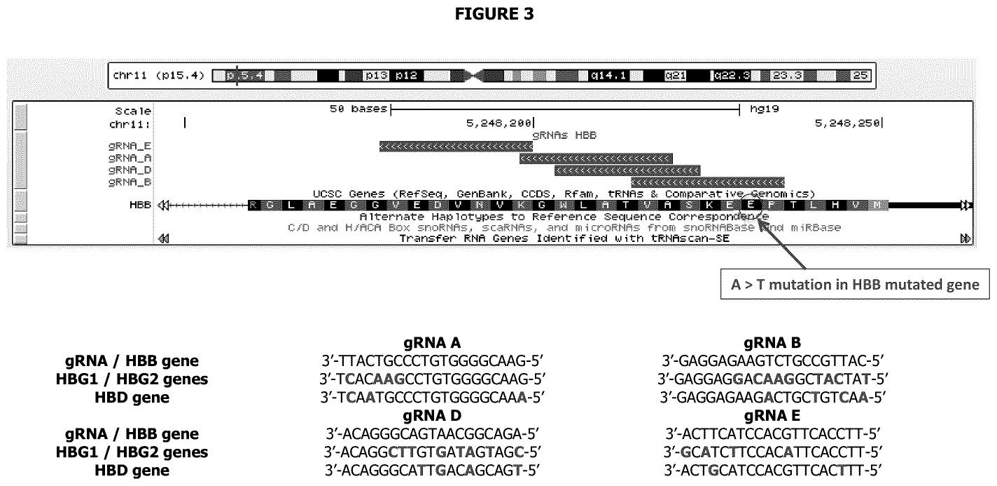

To reduce the expression of the sickle beta-globin gene (i.e. BetaS-globin gene), we selected 4 publicly available gRNAs targeting the exon 1 of the beta-globin gene (Cradick et al., Nucleic Acids Res, 2013, 41(20):9584-92; Liang et al., Protein Cell, 2015, 6(5):363-72) (gRNA spacer-encoding sequences A, B, D and E, FIG. 3, respectively SEQ ID NO: 23 to 26).

Bioinformatic prediction using COSMID (CRISPR Off-target Sites with Mismatches, Insertions, and Deletions; crispr.bme.gatech.edu; Cradick et al, MolTher Nucleic Acids, 2014, 3(12):e214) showed a low number of predicted off-targets, all of them harboring ≥2 mismatches with the delta-globin target sequence (FIG. 3).

Importantly, HBG1/2 genes (coding for gamma-globins) were not included in the list of potential off-targets, the selected gRNAs displaying low similarity with the sequence of gamma-globin genes. Amongst the 4 gRNA spacers, only gRNA spacer E displays less than 3 mismatches with the sequence of exon 1 of the delta-globin gene. Bioinformatic prediction of off-target activity indicates this gene as a potential off-target of gRNA E.

The gRNA-encoding sequences A, B, D and E were cloned in MLM3636 plasmids (MLM3636, Addgene plasmid #43860), generating the following plasmids:
MLM3636 gRNA A coding for gRNA A
MLM3636 gRNA B coding for gRNA B
MLM3636 gRNA D coding for gRNA D
MLM3636 gRNA E coding for gRNA E

For the generation of MLM3636 plasmids carrying the gRNA-encoding sequences A, B, D and E, the following protocol was applied:

a. Annealing gRNA Oligos

### Oligonucleotide Sequences:

| | | SEQ |
|---|---|---|
| Oligo Name | Sequence 5′ to 3′ (*) | ID NO: |
| Oligo FOR-gRNA A | ACACCGCTTGCCCCACAGGGC | 37 |
| | AGTAAG | |
| Oligo REV-gRNA A | AAAACTTACTGCCCTGTGGGG | 38 |
| | CAAGCG | |
| Oligo FOR-gRNA B | ACACCGTAACGGCAGACTTCT | 39 |
| | CCTCG | |
| Oligo REV-gRNA B | AAAACGAGGAGAAGTCTGCCG | 40 |
| | TTACG | |
| Oligo FOR-gRNA D | ACACCGTCTGCCGTTACTGCC | 41 |
| | CTGTG | |
| Oligo REV-gRNA D | AAAACACAGGGCAGTAACGGC | 42 |
| | AGACG | |
| Oligo FOR-gRNA E | ACACCGAAGGTGAACGTGGAT | 43 |
| | GAAGTG | |
| Oligo REV-gRNA E | AAAACACTTCATCCACGTTCA | 44 |
| | CCTTCG | |

(*) In bold: nucleotide sequence encoding the gRNA spacer

Preparation of 10×annealing Buffer [400 μl 1M Tris HCl pH8, 200 μl 1M MgCl2, 100 μl 5M NaCl, 20 μl 0.5M EDTA pH8, 280 μl DEPC-water]. Preparation of MIX 1 for gRNA oligo annealing [1 μl 100 μM gRNA oligo FOR, 1 μl 100 μM gRNA oligo REV, 5 μl 10×annealing Buffer, 43 μl DEPC-water]. Annealing reaction in PCR machine with gradient annealing temperature: from 95° C. to 4° C. in 60 minutes, thus decreasing the annealing temperature of −1.5° C. each minute.

b. Digestion of MLM3636 Plasmid

Incubate the digestion mix reaction [x μl (2.5 μg) of MLM3636 plasmid (Addgene plasmid #43860), 5 μl of BSMB I enzyme (50 U), 5 μl of enzyme buffer 10×, (50−x) μl of DEPC-water]over-night at 55° C. Purify from low melting agarose (0.8%) gel the linearized MLM3636 plasmid (size: 2265 bp) with QIAquick Gel Extraction Kit (QIAGEN).

c. Insertion of gRNA within MLM3636 Plasmid

Incubation of ligation mix [x μl (10 ng) linearized MLM3636 plasmid, 1.1 μl of annealed gRNA-encoding sequence (diluted 1:10), 5 μl of 2×Ligase Buffer, 1 μl of Ligase (QUICK LIGASE NEB-Biolabs-M22OO), (10−x) μl of DEPC-water] for 15 minutes at room temperature.

d. Transformation of Bacteria and Amplification of Plasmid

Chemical competent E. coli bacteria (One Shot TOP10 Chemically competent E. Coli-Invitrogen-C4040) are transformed with 5 μl of ligation products, following manufacter's instruction, and plated in LB AGAR+100 μg/ml Ampicillin over-night at 37° C.

Single-colonies of transformed E. coli bacteria are picked from LB AGAR plate and grown in 3 ml of LB medium+100 μg/ml Ampicillin (inoculation culture) over-night at 37° C. For maxiprep cultures, 0.5 ml of inoculation culture is grown in 250 ml of LB medium+100 μg/ml Ampicillin.

e. Purification of Plasmid DNA

Plasmid DNA is isolated from 250 ml of maxiprep culture of transformed E. coli bacteria by using PureLink HiPure Plasmid DNA Purification Kit (Invitrogen—K2100) applying manufacter's instruction.

2. Selection of gRNAs Targeting β-Globin Gene: Design of Novel gRNAs

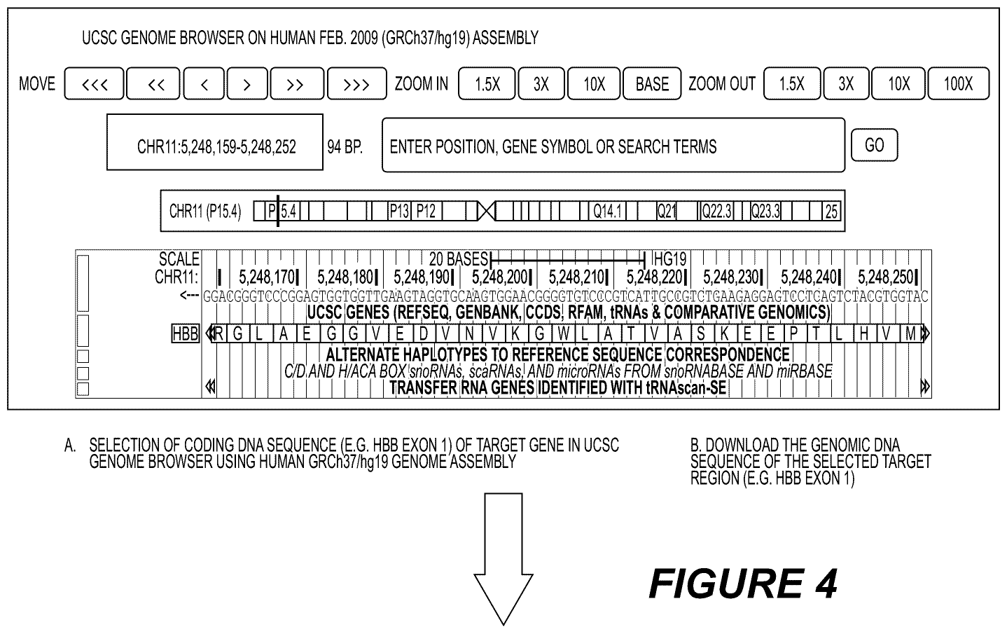

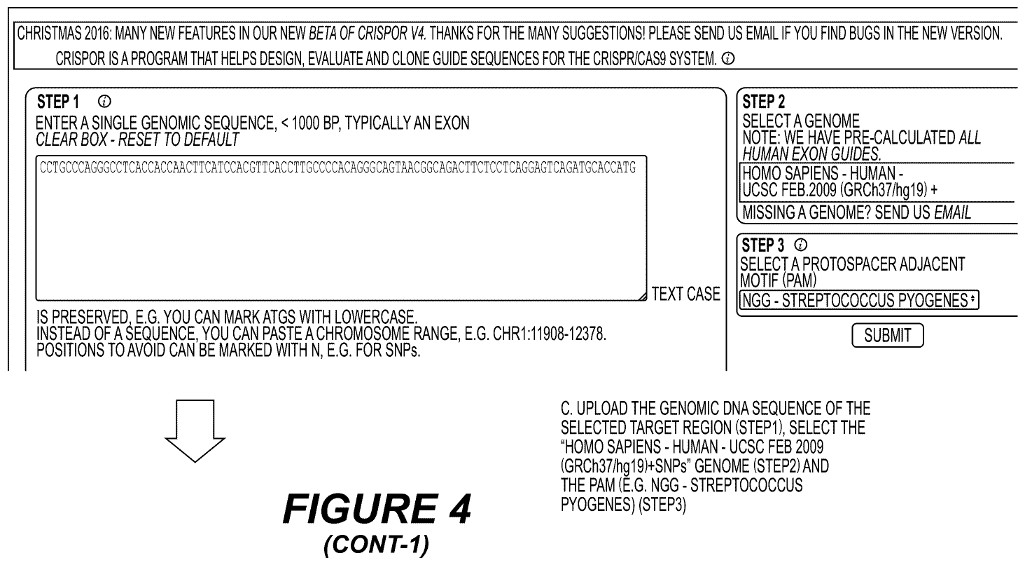

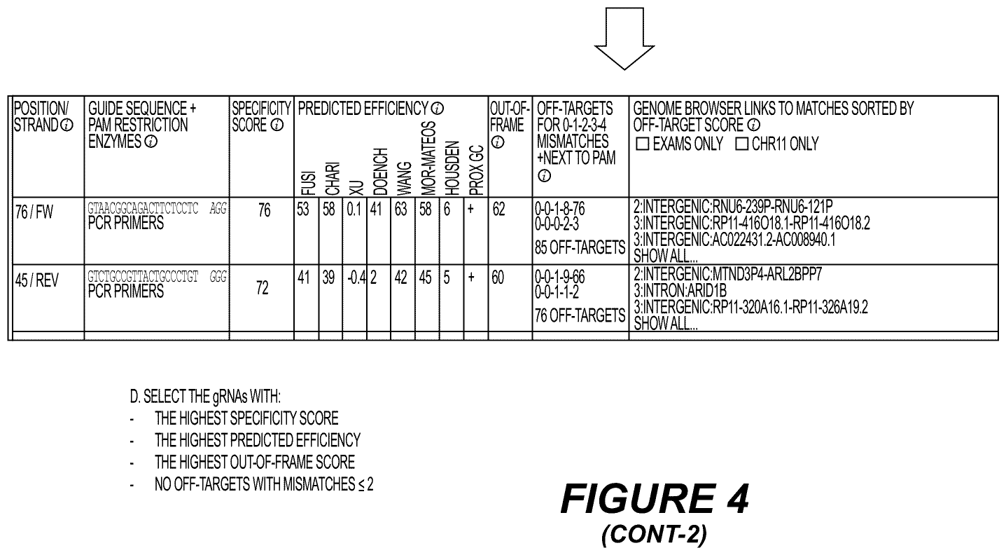

Novel gRNAs spacer-encoding sequences (F, G, H, I, J, K, L, M, N and O—respectively SEQ ID NOs: 27 to 36) were designed by using CRISPOR tool (crispor.tefor.net). The genomic DNA sequence of the target region (e.g. exon 1 or exon 2 of HBB gene) was selected (FIG. 4A) using human GRCh37/hg19 genome assembly and downloaded (FIG. 4B) from UCSC Genome Browser (genome-euro.ucsc.edu/index.html). The genomic DNA sequence of the target region was uploaded on crispor.tefor.net and gRNAs associated with a specific PAM (e.g. NGG—Streptococcus Pyogenes or NGA—S. Pyogenes mutant VQR) were designed based on the “Homo sapiens—human—UCSC February 2009 (GRCh37/hg19)+SNPs” genome (FIG. 4C). From the list of the resulting gRNAs, we selected the gRNAs with a highest (i) specificity score (cfdSpecScore ≥85), (ii) predicted efficiency (ChariEffScore ≥38) and (iii) out-of-frame score (≥60) and no off-targets with mismatches ≥2 in delta- and gamma-globin genes (FIG. 4D).

3. Cleavage Efficiency of gRNAs a, B, D and E in K562 and HUDEP-2 Erythroid Cell Lines

Fetal K562 and adult HUDEP-2 erythroid cells are known to naturally comprise the beta-globin gene in their genome. Therefore, we tested the gRNAs targeting the beta-globin gene in these cell lines.

One million cells were transfected with 4 μg of a Cas9-GFP expressing plasmid (pMJ920, Addgene plasmid #42234) and 0.8 μg of each gRNA-containing plasmid (MLM3636 gRNA A, MLM3636 gRNA B, MLM3636 gRNA D and MLM3636 gRNA E) in a 100 μl volume using Nucleofector I (Lonza). Control cells were treated with 4 μg of a Cas9-GFP expressing plasmid (pMJ920, Addgene plasmid #42234). We used AMAXA Cell Line Nucleofector Kit V (VCA-1003) for K562 and HUDEP-2 (T16 and L-29 programs). After transfection, K562 were maintained in RPMI 1640 medium (Lonza) containing 2 mM glutamine and supplemented with 10% fetal bovine serum (FBS, BioWhittaker, Lonza), HEPES (20 mM, LifeTechnologies), sodium pyruvate (1 mM, LifeTechnologies) and penicillin and streptomycin (100 U/ml each, LifeTechnologies) and HUDEP-2 were maintained as described in Canver et al., Nature, 2015,527(7577):192-7. One week after transfection, DNA was extracted using PURE LINK Genomic DNA Mini kit (LifeTechnologies) following manufacturer's instructions.

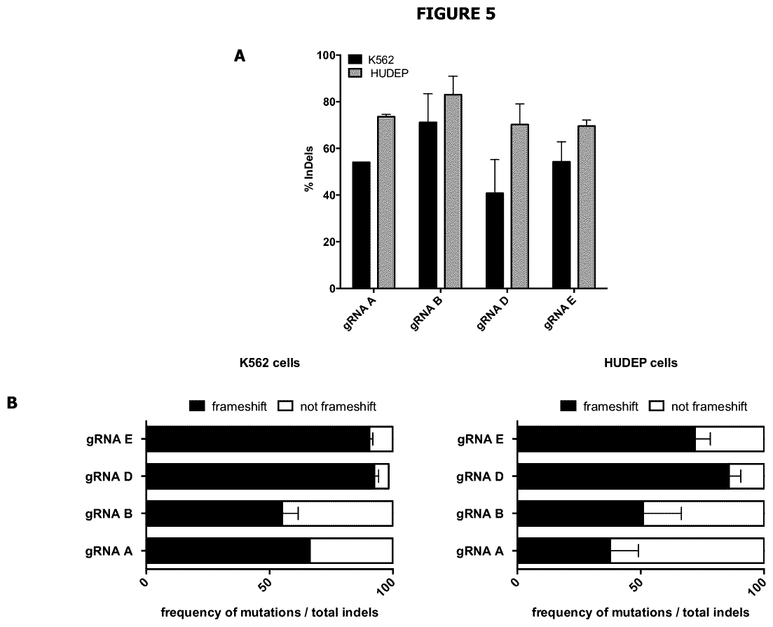

The genomic region of fetal K562 and adult HUDEP-2 erythroid cells encompassing the gRNA target sites was amplified by PCR. PCR was performed using primers HBBex1 F (5′-CAGCATCAGGAGTGGACAGA-3′, SEQ ID NO: 9) and HBBex1 R (5′-AGTCAGGGCAGAGCCATCTA-3′, SEQ ID NO: 10). We performed Sanger sequencing and TIDE analysis to evaluate the frequency of InDels and frameshift mutations. All the screened gRNAs (i.e. A, B, D, E) were able to cut at >35% of the genomic loci in transfected K562 and HUDEP-2 cells (FIG. 5A). The cells transfected with gRNA D led to the highest frequency of frameshift mutations, which resulted in the generation of stop-codons in the exon 1 (FIG. 5B). These results showed that gRNA A, B, D and E are particularly efficient to generate frameshift mutations of beta-globin gene in fetal K562 and adult HUDEP-2 erythroid cells resulting in the generation of stop codon in Exon 1.

### 4. Down-Regulation of Beta-Globin Expression in HUDEP-2

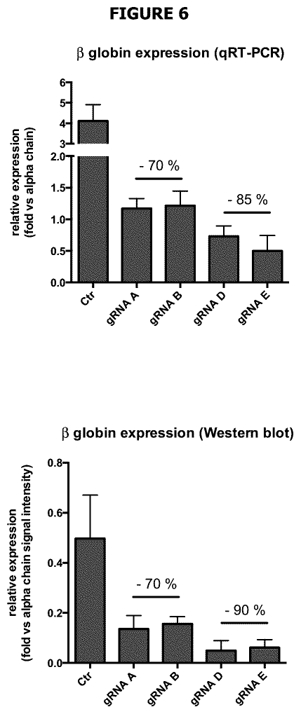

The efficiency of beta-globin knock-down was evaluated in HUDEP2 cells, which express high levels of the beta-globin chain (Kurita et al., PLoS One, 2013, 8(3):e59890). HUDEP-2 cells were transfected with 4 μg of a Cas9-GFP expressing plasmid (pMJ920, Addgene plasmid #42234) and 0.8 μg of each gRNA-containing plasmid (MLM3636 gRNA A, MLM3636 gRNA B, MLM3636 gRNA C and MLM3636 gRNA D), as described above (Example 3). Control cells were treated with 4 μg of a Cas9-GFP expressing plasmid (pMJ920, Addgene plasmid #42234). After one week, total RNA was extracted using RNeasy micro kit (QIAGEN) following manufacturer's instructions. Mature transcripts were reverse-transcribed using SuperScript First-Strand Synthesis System for RT-PCR (Invitrogen) with oligo(dT) primers. qRT-PCR was performed using SYBR green (Applied Biosystems). Primers HBB F (5′-GCAAGGTGAACGTGGATGAAGT-3′, SEQ ID NO: 11) and HBB R (5′-TAACAGCATCAGGAGTGGACAGA-3′, SEQ ID NO: 12) were used to amplify the beta-globin transcripts. Primers HBA1 F (5′-CGGTCAACTTCAAGCTCCTAA-3′, SEQ ID NO: 13) and HBA1 R (5′-ACAGAAGCCAGGAACTTGTC 3′, SEQ ID NO: 14) were used to amplify the alpha-globin transcripts. Beta-globin expression results were normalized to alpha-globin. In parallel, total proteins were extracted in lysis buffer [PBS 1×, 50 mM, TriS-HCl PH 7.4-7.5, 150 mM NaCl, 0.5% DOC, 0.1% SDS, 2 mM EDTA, 1% Triton, protease inhibitor 7× (EDTA-Free Protease Inhibitor Cocktail, Roche) and phosphatase inhibitor 10× (PhosphoSTOP, Roche)], subjected to 3 rounds of sonication (three cycles of 10 pulses, Amplitude 0.7, 0.5 s oscillation) and to 3 freeze/thaw cycles (3 min each). Lysates were centrifuged at 12.000×g for 12 min at 4° C., and supernatants were used for western blot analysis. We measured protein content using the Bradford Protein Assay kit with bovine serum albumin (BSA) as reference standard. After boiling for 5 min in loading buffer (30% glycerol, 5% SDS, 9.25% Dithiothreitol, 1 μl of Bromophenol Blue, Tris-HCl 0.5 M, pH 6.8). samples containing 20-50 μg protein were separated using a 15% acrylamide gel SDS-PAGE electrophoresis. The transfer was performed at 250 mA for 2 hour at 4° C. or room temperature (RT). The PDVF membranes were dried and then incubated in blocking solution TBS-Tween 0.1% (Tris-Buffered Saline+Tween 20; TBS-T; Sigma Aldrich) 5% milk over-night at 4° C., and stained for 1-2 hours at RT with primary antibodies diluted in TBS-Tween 5% milk solution. The primary antibodies are specific for beta-globin (dilution 1:200; hemoglobin beta (37-8), sc-21757, Santa Cruz Biotechnology) and alpha-globin (dilution 1:200; hemoglobin alpha (D-16), sc-31110, Santa Cruz Biotechnology). After 3 washes (10 minutes each) in TBS-Tween, antibody staining was revealed using HRP-conjugated anti-mouse (1:5.000; Thermo Scientific) and HRP-conjugated anti-goat (1:5.000; Thermo Scientific) for 1 hour at RT in TBS-T 5% milk solution. Blots were developed with ECL system (Immobilon Western, Millipore) and were exposed to x-ray films (different exposure times according to the intensity of signals). Membranes were stripped for 15′ with Stripping Buffer (Thermo Scientific). The bands corresponding to beta-globin were quantified by using ImageJ software and/or Gel Pro software and the values (in pixels) obtained were normalized to those of the alpha-globin bands. Both qRT-PCR (FIG. 6) and Western Blot (FIG. 6) analysis showed a reduction in the beta-globin expression in cells treated with Cas9+gRNAs targeting HBB gene, which was more pronounced in cells electroporated in the presence of the gRNAs allowing the highest frequency of frameshift mutations (gRNA D and E).

These results showed that gRNA A, B, D and E are particularly efficient to disrupt the expression of beta-globin in HUDEP-2 cells.

5. Cleavage Efficiency of Selected gRNAs in HSPCs
5.1 Transfection of Primary HSPCs with gRNA B, D and E: Editing Efficiency

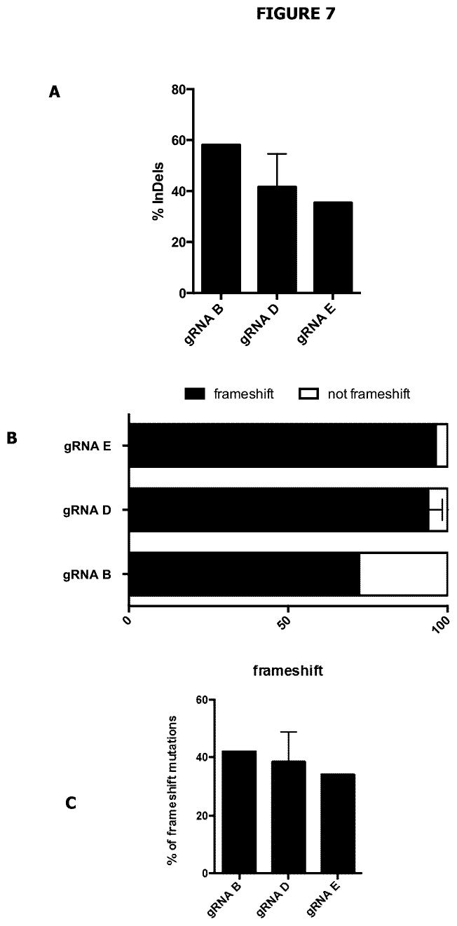

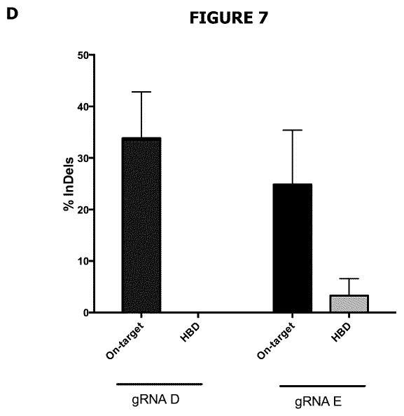

gRNAs allowing the highest frequency of frameshift mutations (B, D and E) were tested in adult HSPC from a healthy donor. HSPC were cultured in expansion medium: StemSpan SFEM medium (StemCell Technologies), containing 2 mM glutamine, penicillin and streptomycin (100 U/ml each, Gibco, LifeTechnologies), Flt3-Ligand (300 ng/ml, Peprotech), SCF (300 ng/ml, Peprotech), TPO (100 ng/ml, Peprotech) and IL3 (60 ng/ml, Peprotech). 48 hours after thawing, one million cells were transfected with 4 μg of a Cas9-GFP expressing plasmid (pMJ920, Addgene plasmid #42234) and 1.6 μl of each gRNA-containing plasmid (MLM3636 gRNA B, MLM3636 gRNA C and MLM3636 gRNA D) in a 100 μl volume using Nucleofector I (Lonza). Control cells were treated with 4 μg of a Cas9-GFP expressing plasmid (pMJ920, Addgene plasmid #42234). We used AMAXA Human CD34 Cell Nucleofector Kit (VPA-1003) for HSPC (U-08 program). After transfection, HSPC were maintained in the same medium supplemented with Z-VAD-FMK (120 uM, InvivoGen) and StemRegenin 1 (750 uM, Stem Cell Technologies). On day 5 after transfection, DNA was extracted to evaluate the editing efficiency, as described above for K562 and HUDEP-2 cells (Example 3). Genome editing efficiency was higher for gRNA B (FIG. 7A), however the rate of frameshift mutations generated by gRNA B was lower compared to gRNA D and E (FIG. 7B). Overall, gRNA B and D allowed the highest absolute frequency of frameshift mutations (FIG. 7C) in HSPC. However, gRNA D was selected for the following experiments, because it generated non-frameshift mutations at a lower frequency (FIG. 7B) and did not have predicted off-targets in the beta-like globin genes.

These results showed that gRNA B, D and E are particularly efficient to generate frameshift mutations of beta-globin gene in HSPC.

### 5.2 Transfection of Primary HSPC Cells: Off Target Analysis

To evaluate off-target activity in primary HSPCs, plasmids encoding the selected gRNAs were individually delivered together with a Cas9-GFP-expressing plasmid to cord blood-derived CD34+ HSPCs. Protocol is slightly different from 5.1. Cells were transfected with 4 μg of Cas9-GFP expressing plasmid and 3.2 μg of each gRNA-containing vector using Nucleofector I (Lonza), AMAXA Human CD34 Cell Nucleofector Kit (VPA-1003) and U08 program. Transfection efficiency was verified by flow cytometry analyses 18 hours after electroporation (30-50% of GFP+ Cas9-expressing cells).

TIDE (Tracking of Indels by Decomposition) analysis (Brinkman E K et al., 2014) of the genomic region containing HBB exon 1 and amplified from genomic DNA extracted 4 days after transfection showed that gRNA D and E display a cleavage efficiency of ≈35% and ≈25%, respectively, with a frequency of frameshift mutations of 90-95% for both the gRNAs (not shown). Conversely, gRNA B displays an editing efficiency of ≈60% with a lower frequency of frameshift mutations in comparison with gRNA D and E (not shown). TIDE analysis the genomic region containing HBD exon 1 showed absence of InDels in samples treated with gRNA D, whereas ≈3% of HBD alleles are edited (“off-target”) upon treatment with gRNA E (FIG. 7D). This result can be explained by the low number of mismatches (2) between gRNA E sequence and the corresponding off-target in HBD exon 1 (FIG. 3), whereas a higher number of mismatches is observed for gRNA D (4; FIG. 3), which likely decreases the probability of off-target activity in the HBD gene.

### 6. Down-Regulation of Beta-Globin Expression in HSPC-Derived Erythroid Cells

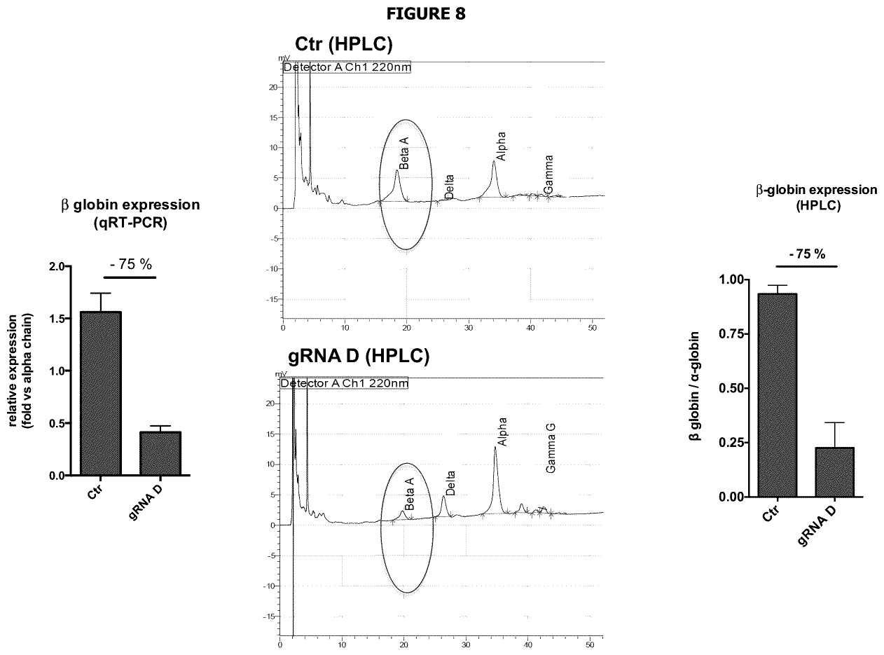

Cas9 and gRNA D were delivered by plasmid transfection in adult HSPC derived from a healthy donor (plasmids pMJ920 Cas9-GFP and MLM3636 gRNA D) as described above (Example 5). Control cells were electroporated in the presence of the plasmid pMJ920. One day after, GFP-positive HSPC were sorted by FACS 2 days after transfection, HSPC were differentiated towards the erythroid lineage in liquid culture as previously described (Sankaran, Science, 2008, 322(5909):1839-42). After 11 days, RNA was extracted from mature erythroid cells to evaluate the beta-globin expression levels. Total RNA was extracted using RNeasy micro kit (QIAGEN) following manufacturer's instructions. Mature transcripts were reverse-transcribed using SuperScript First-Strand Synthesis System for RT-PCR (Invitrogen) with oligo(dT) primers. qRT-PCR was performed using SYBR green (Applied Biosystems). Primers HBB F (5′-GCAAGGTGAACGTGGATGAAGT-3′, SEQ ID NO: 11) and HBB R (5′-TAACAGCATCAGGAGTGGACAGA-3′, SEQ ID NO: 12) were used to amplify the beta-globin transcripts. Primers HBA1 F (5′-CGGTCAACTTCAAGCTCCTAA-3′, SEQ ID NO: 13) and HBA1 R (5′-ACAGAAGCCAGGAACTTGTC 3′, SEQ ID NO: 14) were used to amplify the alpha-globin transcripts. Beta-globin expression results were normalized to alpha-globin. In parallel, reverse phase HPLC (RP-HPLC) analysis of globin chains was performed using a NexeraX2 SIL-30AC chromatograph (Shimadzu) and the LC Solution software. Globin chains from in vitro differentiated mature erythroblasts were separated by HPLC using a 250×4.6 mm, 3.6 μm Aeris Widepore column (Phenomenex). Samples were eluted with a gradient mixture of solution A (water/acetonitrile/trifluoroacetic acid, 95:5:0.1) and solution B (water/acetonitrile/trifluoroacetic acid, 5:95:0.1). The absorbance was measured at 220 nm. Both qRT-PCR and RP-HPLC analyses showed a dramatic down-regulation of beta-globin expression in mature erythroblasts electroporated with plasmid MLM3636 gRNA D (FIG. 8).

These results showed that gRNA D is particularly efficient to disrupt the expression of beta-globin in HSPC-derived erythroblasts.

### Example 4: Optimization of gRNA Activity

The original gRNA scaffold developed by Cong et al., Science, 2013,339(6121):819-23 was recently optimized by Dang et al., Genome Biol, 2015, 16:280 to increase knock-out efficiency.

The gRNA spacer-encoding sequences B, D and E (respectively SEQ ID NOs: 24, 25 and 26) were cloned in Dang p.hU6 gRNA plasmids (Addgene #53188), generating the following plasmids:
Dang p.hU6 gRNA B coding for gRNA B
Dang p.hU6 gRNA D coding for gRNA D
Dang p.hU6 gRNA E coding for gRNA E

For the generation of Dang p.hU6 plasmids (Addgene #53188) carrying gRNA B, D and E, the following protocol was applied:

a. Annealing gRNA Oligos

### Oligonucleotide Sequences:

| | | SEQ |
|---|---|---|
| Oligo Name | Sequence 5′ to 3′ (*) | ID No: |
| Oligo FOR-Opt_gRNA B | CACCGTAACGGCAGACTTCTC | 15 |
| | CTC | |
| Oligo REV-Opt_gRNA B | AAACGAGGAGAAGTCTGCCGT | 16 |
| | TAC | |
| Oligo FOR-Opt_gRNA D | CACCGTCTGCCGTTACTGCCC | 17 |
| | TGT | |
| Oligo REV-Opt_gRNA D | AAACACAGGGCAGTAACGGCA | 18 |
| | GAC | |
| Oligo FOR-Opt_gRNA E | CACCGAAGGTGAACGTGGATG | 19 |
| | AAGT | |
| Oligo REV-Opt_gRNA E | AAACACTTCATCCACGTTCAC | 20 |
| | CTTC | |

(*) In bold: nucleotide sequence encoding the gRNA spacer

Preparation of MIX 1 for gRNA oligo annealing [8 μl 10 μM gRNA oligo FOR-Opt, 8 μl 10 μM gRNA oligo REV-Opt, 2 μl 10×NEB Ligase buffer (Biolabs—M22OO), 2 μl DEPC-water]. Annealing reaction in PCR machine, following this PCR program: from 96° C. 300 seconds, 85° C. 20 seconds, 75° C. 20 seconds, 65° C. 20 seconds, 55° C. 20 seconds, 45° C. 20 seconds, 35° C. 20 seconds, 25° C. 20 seconds

b. Digestion of Dang p.hU6 Plasmid

Incubate the digestion mix reaction [x μl (20 μg) of Dang p.hU6 plasmid (Addgene #53188), 10 μl of BbsI enzyme (100 U), 10 μl of enzyme buffer 10×, (100−x) μl of DEPC-water]over-night at 37° C. Purify from low melting agarose (0.8%) gel the linearized Dang p.hU6 plasmid (size: 3515 bp) with QIAquick Gel Extraction Kit (QIAGEN).

c. Insertion of gRNA within Dana p.hU6 Plasmid

Incubation of ligation mix [x μl (50 ng) linearized MA128.hU6 plasmid, 1 μl of annealed gRNA oligos, 1 μl of 10×Ligase Buffer, 1 μl of Ligase (QUICK LIGASE NEB—M22OO), (10−x) μl of DEPC-water] for 15 minutes at room temperature.

d. Transformation of Bacteria and Amplification of Plasmid

Chemical competent E, co/i bacteria (One Shot TOP10 Chemically competent E. Coli—Invitrogen—C4040) are transformed with 5 μl of ligation products, following manufacter's instruction, and plated in LB AGAR+100 μg/ml Ampicillin over-night at 37° C.

Single-colonies of transformed E. coli bacteria are picked from LB AGAR plate and grown in 3 ml of LB medium+100 μg/ml Ampicillin (inoculation culture) over-night at 37° C. For maxiprep cultures, 0.5 ml of inoculation culture is grown in 250 ml of LB medium+100 μg/ml Ampicillin.

e. Purification of Plasmid DNA

Plasmid DNA is isolated from 250 ml of maxiprep culture of transformed E. coli bacteria by using PureLink HiPure Plasmid DNA Purification Kit (Invitrogen—K2100) applying manufacter's instruction.

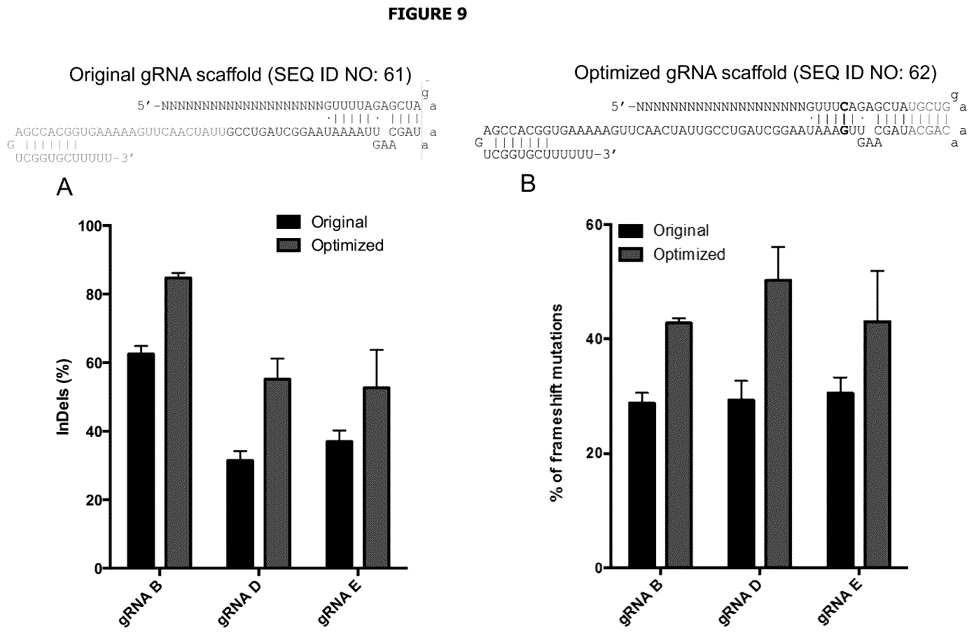

One million of K562 cells were transfected with 4 μg of a Cas9-GFP expressing plasmid (pMJ920, Addgene plasmid #42234) and 0.8 of each gRNA-containing plasmid (MLM3636 gRNA B, MLM3636 gRNA C and MLM3636 gRNA D, Dang p.hU6 gRNA B, Dang p.hU6 gRNA C and Dang p.hU6 gRNA D) in a 100 μl volume using Nucleofector I (Lonza). Control cells were treated with 4 μg of a Cas9-GFP expressing plasmid (pMJ920, Addgene plasmid #42234). We used AMAXA Cell Line Nucleofector Kit V (VCA-1003) for K562 cells (T16 program). After transfection, K562 were maintained in RPMI 1640 medium (Lonza) containing 2 mM glutamine and supplemented with 10% fetal bovine serum (FBS, BioWhittaker, Lonza), HEPES (20 mM, LifeTechnologies), sodium pyruvate (1 mM, LifeTechnologies) and penicillin and streptomycin (100 U/ml each, LifeTechnologies). One week after transfection, DNA was extracted using PURE LINK Genomic DNA Mini kit (LifeTechnologies) following manufacturer's instructions. All the gRNAs with the optimized structure (Dang p.hU6 gRNA B, Dang p.hU6 gRNA C and Dang p.hU6 gRNA D; Dang et al., Genome Biol, 2015, 16:280) show higher InDels efficiency (FIG. 9A) and frequency of frameshift mutation in HBB gene (FIG. 9B) compared to the corresponding gRNAs with original structure (MLM3636 gRNA B, MLM3636 gRNA C and MLM3636 gRNA D; Cong et al., Science, 2013,339(6121):819-23).

These results showed that the modification of the scaffold in the gRNAs targeting the beta-globin gene (see Example 3) can further increase their frequency of gene disruption.

### Example 5: Construction of a Recombinant Viral Vector (i.e. Lentivector) According to the Invention

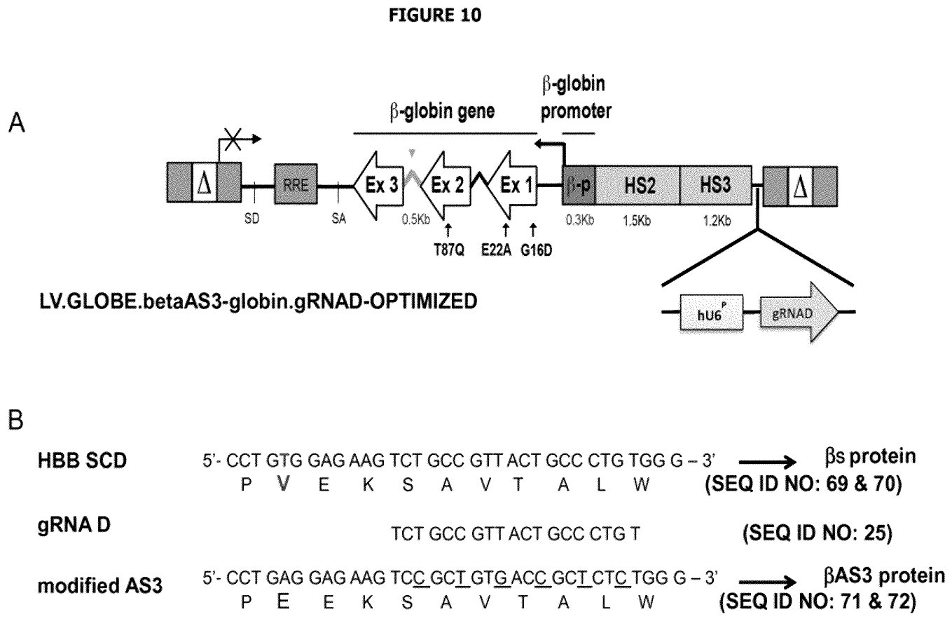

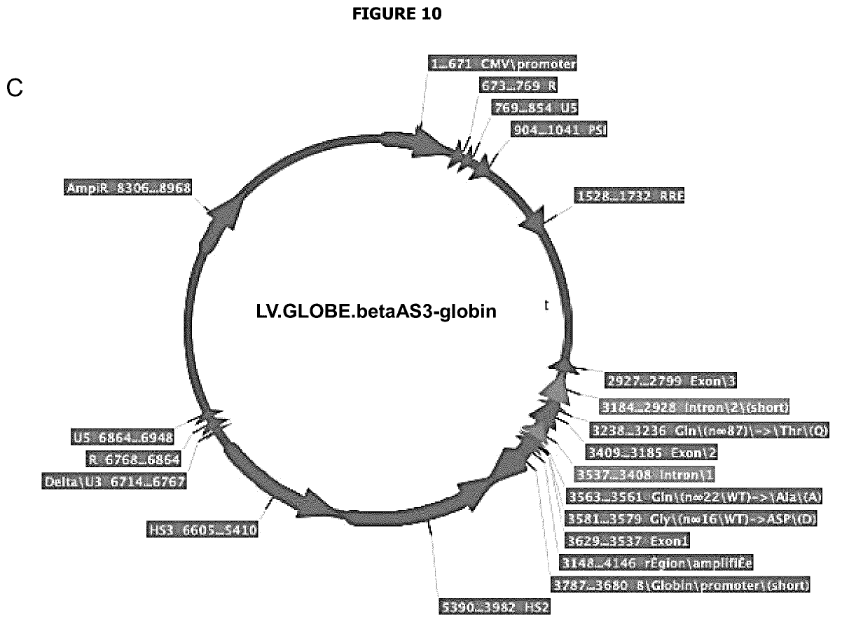

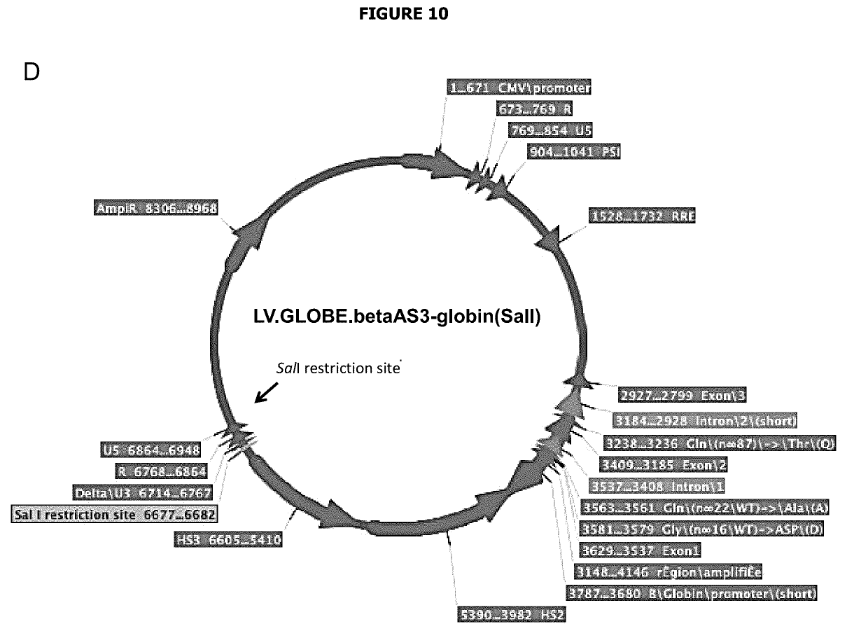

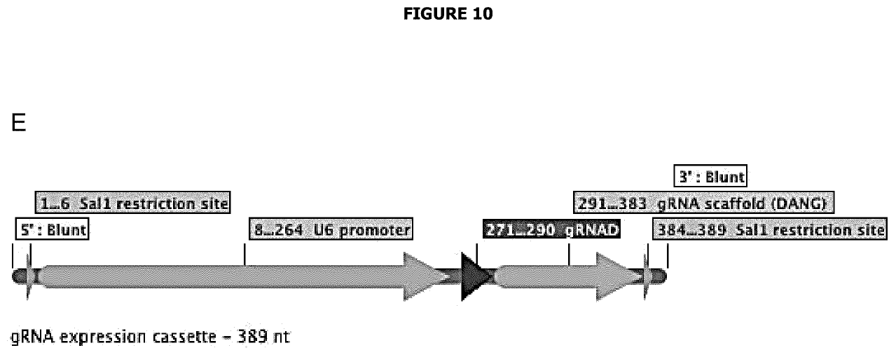

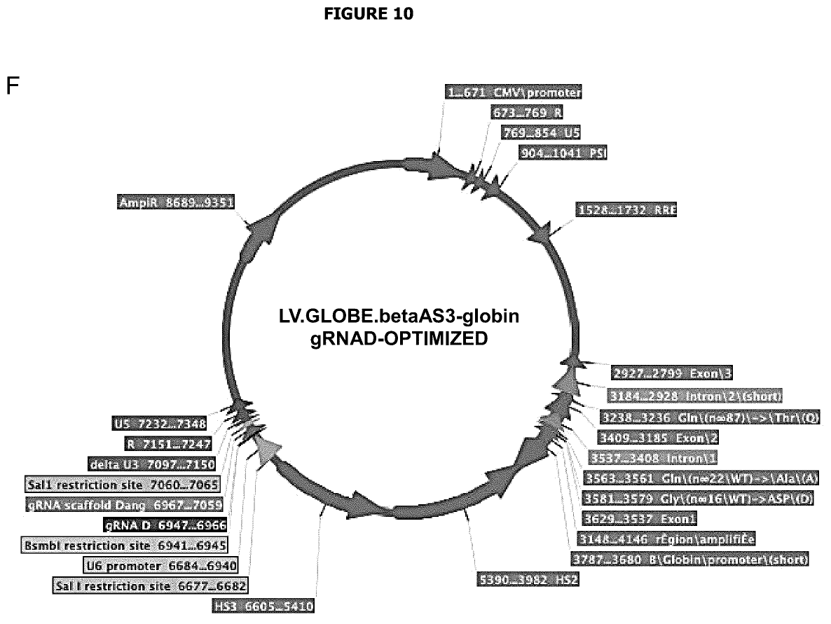

The LV.GLOBE.betaAS3-globin.gRNA D-OPTIMIZED lentiviral construct (FIG. 10A, such as SEQ ID NO: 47) carries: (1) an anti-sickling gene (FIG. 10B, e.g. modified Beta AS3 SEQ ID NO: 8) harboring silent mutations (indicated as underscored letters in FIG. 10B) inserted by site-directed mutagenesis in order to impair the gRNA binding to the transgene and the three antisickling mutations [Gly16Asp (G16D), Glu22Ala (E22A) and Thr87Gln (T87Q)] in the exons 1 and 2 (FIG. 10A); (2) a gRNA showing (i) a high efficiency of beta-globin gene disruption; (ii) a high rate of frameshift mutations; (iii) a low off-target activity (e.g. no off-targets in the beta like-globin genes), such as gRNA D (FIG. 10B), under the control of the human U6 promoter (FIG. 10A).

In FIG. 10C, 10D, 10E and 10F, (A.) the restriction site SalI is inserted between HS3 and DeltaU3 elements of the LV.GLOBE.betaAS3-globin plasmid (FIG. 10C; SEQ ID NO: 45) by site-directed mutagenesis to generate the LV.GLOBE. betaAS3-globin (SalI) plasmid (SEQ ID NO: 46). (B.) A DNA fragment containing the hU6 promoter and the gRNA-encoding sequence (e.g. gRNA D) flanked by SalI restriction sites (called “gRNA expression cassette”; FIG. 10E) is synthesized. (C.) LV.GLOBE. betaAS3-globin (SalI) plasmid (SEQ ID NO: 46) is digested [digestion mix reaction: x μl (20 μg) of LV.GLOBE. betaAS3-globin (SalI) plasmid (SEQ ID NO: 46), 10 μl of SalI enzyme (100 U), 10 μl of enzyme buffer 10×, (100−x) μl of DEPC-water]over-night at 37° C. The linearized LV.GLOBE. betaAS3-globin-globin(SalI) plasmid (size: 10195 bp) is purified by low melting agarose (0.8%) gel using QIAquick Gel Extraction Kit (QIAGEN). In parallel, the gRNA expression cassette is digested [digestion mix reaction: x μl (20 μg) of gRNA expression cassette, 10 μl of SalI enzyme (100 U), 10 μl of enzyme buffer 10×, (100−x) μl of DEPC-water]over-night at 37° C. The linearized gRNA expression cassette (size: 383 bp) is purified by low melting agarose (1.5%) gel using QIAquick Gel Extraction Kit (QIAGEN). (D.) The gRNA expression cassette is inserted within LV.GLOBE. betaAS3-globin-globin(SalI) plasmid through incubation of ligation mix [x μl (50 ng) linearized gRNA expression cassette, γ μl (50 ng) linearized LV.GLOBE. betaAS3-globin-globin(SalI) plasmid, 1 μl of 10×Ligase Buffer, 1 μl of Ligase (QUICK LIGASE NEB—M2200), (10−x−y) μl of DEPC-water] for 15 minutes at room temperature. Chemical competent E. coli bacteria (One Shot TOP10 Chemically competent E. Coli—Invitrogen—C4040) are transformed with 5 μl of ligation products, following manufacter's instruction, and plated in LB AGAR+100 μg/ml Ampicillin over-night at 32° C. Single-colonies of transformed E. coli bacteria are picked from LB AGAR plate and grown in 50 ml of LB medium+100 μg/ml Ampicillin (miniprep cultures) over-night at 32° C. Plasmid DNA is isolated from 10 ml of miniprep culture of transformed E. coli bacteria by using PureLink HiPure Plasmid DNA Purification Kit (Invitrogen—K2100) applying manufacter's instruction. Plasmid DNA will be analyse by Sanger-sequencing to verify that gRNA expression cassette is inserted in the opposite orientation compare to betaAS3-globin expression cassette. Miniprep cultures (10 ml) derived from colonies containing plasmids fitting these criteria are grown in 250 ml of LB medium+100 μg/ml Ampicillin over-night at 32° C. Plasmid DNA is isolated from 250 ml of maxiprep culture of transformed E. coli bacteria by using PureLink HiPure Plasmid DNA Purification Kit (Invitrogen—K2100) applying manufacter's instruction. The isolated plasmid DNA (LV.GLOBE.betaAS3-globin.gRNA D-OPTIMIZED; FIG. 10F, SEQ ID NO: 47) is used as backbone for recombinant lentiviral vector production.

### Example 6: Transduction of HSPC with a Recombinant Lentiviral Vector According to the Invention and Introduction of Cas9 into the Transduced Cell

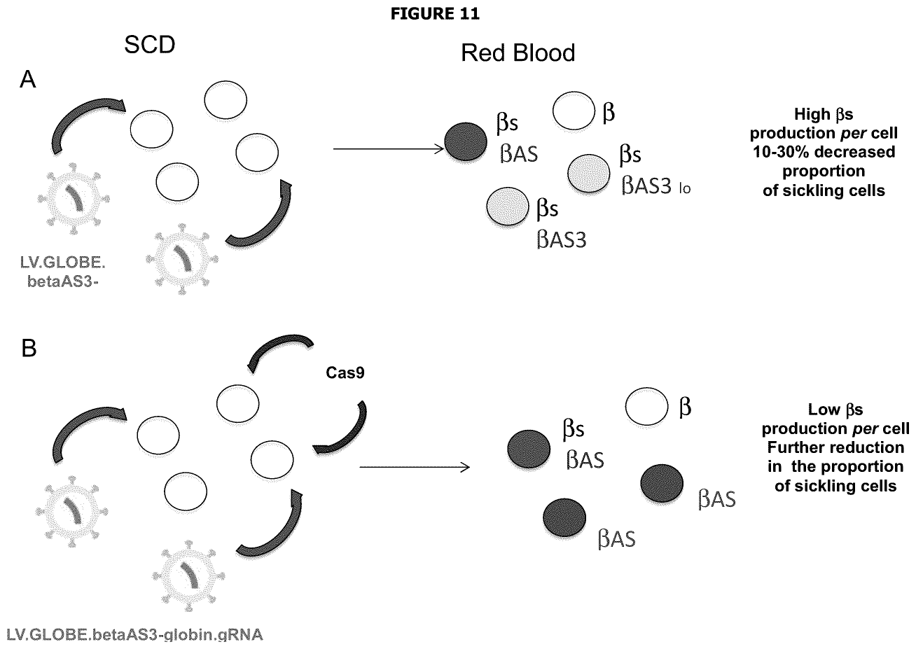

(A) In the classical gene therapy approach the lentiviral vector expressing an anti-sickling gene (e.g. LV.GLOBE.beta-globin and LV.AS3 (Romero et al., JCI, 2016)) does not strongly reduce the sickle beta-globin expression in the erythroid progeny of SCD HSPC and allows the correction of only 10% to 30% of mature Red Blood Cells (FIG. 11A).

(B) SCD HSPC are transduced with the gamma-beta hybrid globin and gRNA expressing lentiviral vector (e.g. LV.GLOBE.gamma-beta-globin.gRNA) and Cas9 is delivered transiently. This approach allows the expression of an anti-sickling transgene and the concomitant reduction of the sickle beta-globin levels, which will lead to an increase frequency of corrected Red Blood Cells Importantly, Cas9-mediated disruption of the sickle beta-globin gene will be observed only in transduced SCD cells where the knock out of the sickle beta-globin is compensated by the expression of the anti-sickling gene, thus avoiding an absence of Beta like chain leading to the risk of alpha-chain precipitation, leading to cell death and anemia, as observed in beta-thalassemia (FIG. 11B).

### Example 7: Genetic Modification of Patient SCD HSPC In Vitro

SCD CD34⁺ HSPC are transduced with lentiviral vectors expressing an anti-sickling gene and a gRNA targeting the beta-globin gene (e.g. LV.GLOBE.betaAS3-globin.gRNAD-OPTIMIZED, SEQ ID NO: 47 or LV.GLOBE-AS3modified.gRNAD, SEQ ID NO: 94) or the intronic erythroid-specific BCL11A enhancer (e.g. LV.GLOBE-AS3modified.gRNA-BCL11Aenhancer, SEQ ID NO: 75) or the gamma-globin promoters (e.g. LV.GLOBE-AS3modified.gRNA-13 bp-del, SEQ ID NO: 76) and Cas9 is delivered transiently (DNA-, RNA-, protein- or lentiviral-delivery).

HSPC derived from bone marrow or mobilized peripheral blood of SCD patients are cultured in RetroNectin (20 μg/ml, Takara Shuzo Co.)-coated plates in expansion medium (pre-activation step): StemSpan SFEM medium (StemCell Technologies), containing 2 mM glutamine, penicillin and streptomycin (100 U/ml each, Gibco, LifeTechnologies), Flt3-Ligand (300 ng/ml, Peprotech), SCF (300 ng/ml, Peprotech), TPO (100 ng/ml, Peprotech) and IL3 (60 ng/ml, Peprotech). 24 hours after thawing (day1), 200.000 cells are transduced with LV.GLOBE.betaAS3-globin.gRNAD-OPTIMIZED (SEQ ID NO: 47) (MOI 20-100) in expansion medium+protein sulfate (4 μg/ml) and plated in RetroNectin (20 μg/ml, Takara Shuzo Co.)-coated 96-well plates. Control cells are transduced with LV.GLOBE. betaAS3-globin. (SalI) (SEQ ID NO: 46) (MOI 20-100) or LV.GLOBE.gRNAD (MOI 20-100) (LV.GLOBE vector carrying gRNA expression cassette without beta AS3 globin transgene). Medium is change 24 hours after transduction (day2) and 1-3*10⁶ cells are transfected with 20 μg of Cas9 mRNA modified with pseudouridine and 5-methylcytidine to reduce immune stimulation (Trilink, #L-6125) in a 100 μl volume using Nucleofector 4D (Lonza). Alternatively, 1-3*10⁵ cells are transfected with 30-180 Cas9 pmol in a 20 μl volume using Nucleofector 4D (Lonza). We use AMAXA Human CD34 Cell Nucleofector Kit (VPA-1003) for HSPC (CA137 program). After transfection, HSPC were maintained in the same medium supplemented with Z-VAD-FMK (120 uM, InvivoGen) and StemRegenin 1 (750 uM, Stem Cell Technologies). The day after (day3), treated HSPC are either in vitro differentiated towards the erythroid lineage using a 3-phase liquid erythroid culture system (Giarratana et al., Blood, 2011,118(19):5071-9) or plated in a semi-solid medium containing cytokines supporting the growth of erythroid and myeloid hematopoietic progenitors (Clonal progenitor assay; medium GFH4435, Stem Cell Technologies). On day 13 of liquid culture and clonal progenitor assay, samples are collected for DNA extraction to evaluate the editing efficiency, as described above for K562 and HUDEP-2 cells (example 3), and the frequency of transduced cells in bulk (erythroid) and clonal culture by PCR followed by Tracking of In/Dels by Decomposition (Brinkman E K, Chen T, Amendola M, and van Steensel B. Easy quantitative assessment of genome editing by sequence trace decomposition. Nucleic acids research. 2014; 42(22):e168.) also called TIDE analysis (as described in example 3) and qPCR (using primers recognizing specifically the lentiviral vector; Miccio et al., Proc Natl Acad Sci USA, 2008,105(30):10547-52), respectively.

A genome-wide analysis of Double Strand Breaks using Genome-wide, unbiased identification of DSBs enabled by sequencing, also called GUIDE-seq (Tsai et al., Nat Biotechnol, 2015, 33(2):187-97) is performed to detect and quantify off-target cleavage sites in HSPC and their differentiated progeny (DNA extracted from samples collected at day13 of clonal progenitor assay). LV integration sites in SCD HSPC are analyzed in order to evaluate the potential genotoxic risk of globin-expressing LV vectors. Integration sites are amplified by ligation-mediated PCR, sequenced and mapped to the human genome, as previously described (Romano et al., Sci Rep, 2016, 6:24724). The anti-sickling globin and betaS-globin expression are evaluated by qRT-PCR in samples collected upon 13, 16, 18 and 21 days of liquid culture differentiation. Total RNA is extracted using RNeasy micro kit (QIAGEN) following manufacturer's instructions. Mature transcripts are reverse-transcribed using SuperScript First-Strand Synthesis System for RT-PCR (Invitrogen) with oligo(dT) primers. qRT-PCR was performed using SYBR green (Applied Biosystems). Primers HBB F (5′-GCAAGGTGAACGTGGATGAAGT-3′, SEQ ID NO: 11) and HBB R (5′-TAACAGCATCAGGAGTGGACAGA-3′, SEQ ID NO: 12) are used to amplify the beta-globin transcripts and primers HBB-AS3 F (5′-AAGGGCACCTTTGCCCAG-3′, SEQ ID NO: 21) and HBB-AS3 R (5′-GCCACCACTTTCTGATAGGCAG-3′, SEQ ID NO: 22) are used to amplify the beta AS3 globin transcripts. Primers HBA1 F (5′-CGGTCAACTTCAAGCTCCTAA-3′, SEQ ID NO: 13) and HBA1 R (5′-ACAGAAGCCAGGAACTTGTC 3′, SEQ ID NO: 14) are used to amplify the alpha-globin transcripts. Beta-globin expression results are normalized to alpha-globin. In parallel, reverse phase HPLC (RP-HPLC) analysis is performed (as described above in Example 6) in genetically modified HSPC differentiated in vitro into fully mature, enucleated Red Blood Cells (day 21 of liquid culture differentiation). The recovery of functional RBC properties is assessed enucleated Red Blood Cells (day 21 of liquid culture differentiation) by evaluating the reversion of the sickling and the correction of the increased adhesiveness and rigidity of SCD cells, features involved in the pathological occurrence of vaso-occlusive events (Picot et al., Am J Hematol, 2015, 90(4):339-45). Sickling dynamics is evaluated in enucleated Red Blood Cells (day 21 of liquid culture differentiation) exposing the cells to an oxygen-deprived atmosphere (0% O₂). Time-course of sickling is monitored in real-time by video microscopy for 1 hour, capturing images every 5 minutes using the AxioObserver Z1 microscope (Zeiss) and a 40×objective.

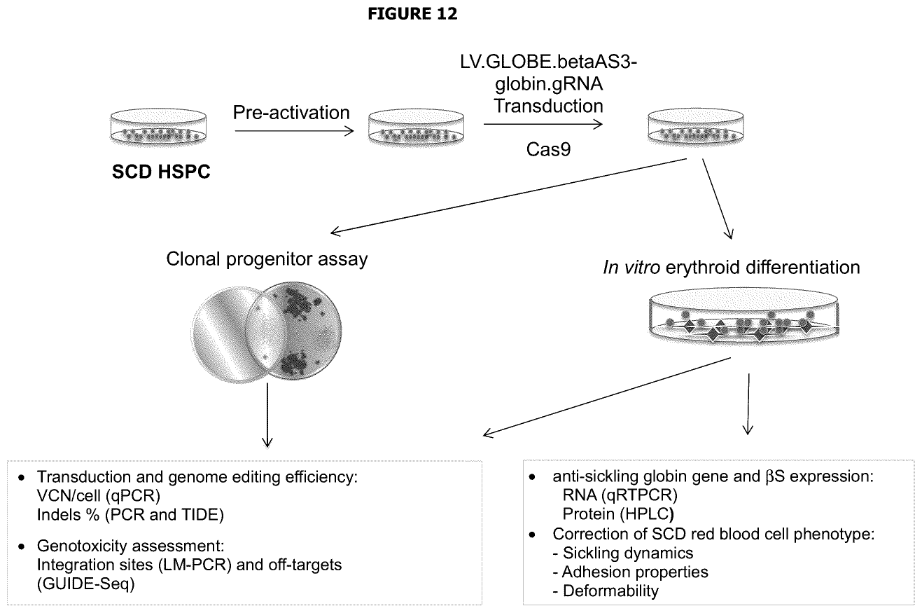

This process is illustrated in FIG. 12.

Such method is applied mutatis mutandis when using any of lentiral vectors of the invention.

### Example 8: Genetic Modification of Patient SCD HSC In Vivo

The engraftment capability of genetically modified patient SCD HSC and the efficacy of the therapeutic approach in Red Blood Cells derived from engrafting SCD HSC are assessed in in vivo mouse experiments. The in vivo frequency of modified HSC and the efficacy of the therapeutic strategy have to be similar to the same parameters measured in vitro in HSPC to exclude any HSC impairment due to our treatment.

HSPC derived from bone marrow or mobilized peripheral blood of SCD patients are cultured in RetroNectin (20 μg/ml, Takara Shuzo Co.)-coated plates in expansion medium (pre-activation step): StemSpan SFEM medium (StemCell Technologies), containing 2 mM glutamine, penicillin and streptomycin (100 U/ml each, Gibco, LifeTechnologies), Flt3-Ligand (300 ng/ml, Peprotech), SCF (300 ng/ml, Peprotech), TPO (100 ng/ml, Peprotech) and IL3 (60 ng/ml, Peprotech). 24 hours after thawing (day1), 1-2*10⁶ cells are transduced with a lentiviral vector expressing an anti-sickling gene and a gRNA targeting the beta-globin gene (e.g. LV.GLOBE.betaAS3-globin.gRNAD-OPTIMIZED, SEQ ID NO: 47 or LV.GLOBE-AS3modified.gRNAD, SEQ ID NO: 94) or a gRNA targeting the intronic erythroid-specific BCL11A enhancer (LV.GLOBE-AS3modified.gRNA-BCL11Aenhancer, SEQ ID NO: 75) or a gRNA targeting the gamma-globin promoters (LV.GLOBE-AS3modified.gRNA-13 bp-del, SEQ ID NO: 76) (MOI 20-100) in expansion medium+protein sulfate (4 μg/ml) and plated in RetroNectin (20 g/ml, Takara Shuzo Co.)-coated 96-well plates. Control cells are transduced with LV.GLOBE.gamma-beta-globin(SalI) (MOI 20-100) and LV.GLOBE.gRNAD (MOI 20-100) (LV.GLOBE vector carrying gRNA expression cassette without beta AS3 globin transgene). Medium is change 24 hours after transduction (day2) and 1-3*10⁶ cells are transfected with 20 μg of Cas9 mRNA modified with pseudouridine and 5-methylcytidine to reduce immune stimulation (Trilink, #L-6125) in a 100 μl volume using Nucleofector 4D (Lonza). Alternatively, 1-3*10⁵ cells are transfected with 30-180 Cas9 pmol in a 20 μl volume using Nucleofector 4D (Lonza). We use AMAXA Human CD34 Cell Nucleofector Kit (VPA-1003) for HSPC (CA137 program). After transfection, HSPC were maintained in the same medium supplemented with Z-VAD-FMK (120 uM, InvivoGen) and StemRegenin 1 (750 uM, Stem Cell Technologies). The day after (day3), cells are injected (0.5-1*10⁶ cells per mouse) i.v. in 9 to 10-week-old partially myeloablated immunodeficient NSG (NOD SCID GAMMA; NOD.Cg-Prkdc^{6cid} Il2rg^{tm1Wjl}/SzJ) mice. After 16 weeks, mice are euthanized and bone marrow, thymus and spleen are analyzed for engraftment of human cells by flow cytometry using anti-human CD45 vs. anti-murine CD45 antibodies. The percentage of engrafted human cells is defined as follows: % huCD45+/(% huCD45++% muCD45+). Analysis of the different hematopoietic cell types present was performed by cell-specific staining for human CD34, human CD45, human CD19, human CD33, human CD71, human CD36 and human CD235a. Transduction efficiency and genome editing efficiency is determined in the purified HSPC and lymphoid and myeloid progeny, as described above in example 7.

Human CD34+ HSPC is isolated from bone marrow of engrafted mice using immunomagnetic separation (CD34 MicroBeads kit human; Miltenyi Biotech). The hCD34-positive fraction is cultured in 3-phase liquid erythroid culture system (Giarratana et al., Blood, 2011,118(19):5071-9) or plated in a semi-solid medium containing cytokines supporting the growth of erythroid and myeloid hematopoietic progenitors (Clonal progenitor assay; medium GFH4435, Stem Cell Technologies). Given the low number of erythroid cells obtained in vivo in NSG mice, the expression of the anti-sickling transgene, the down-regulation of sickle beta-globin expression and the functional correction of the SCD phenotype are assessed ex vivo in the erythroid progeny of modified SCID-Repopulating cells, as describe above (example 7).

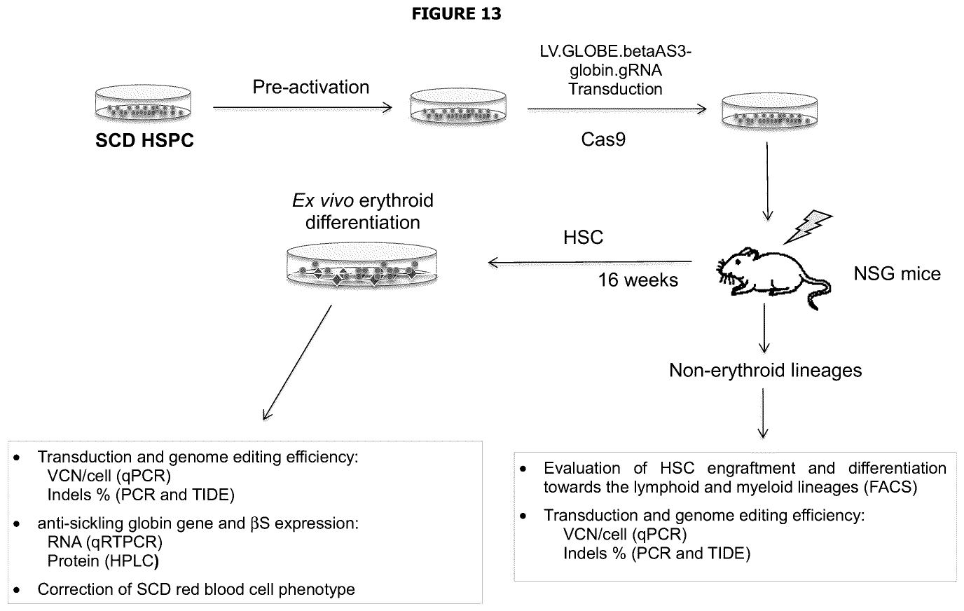

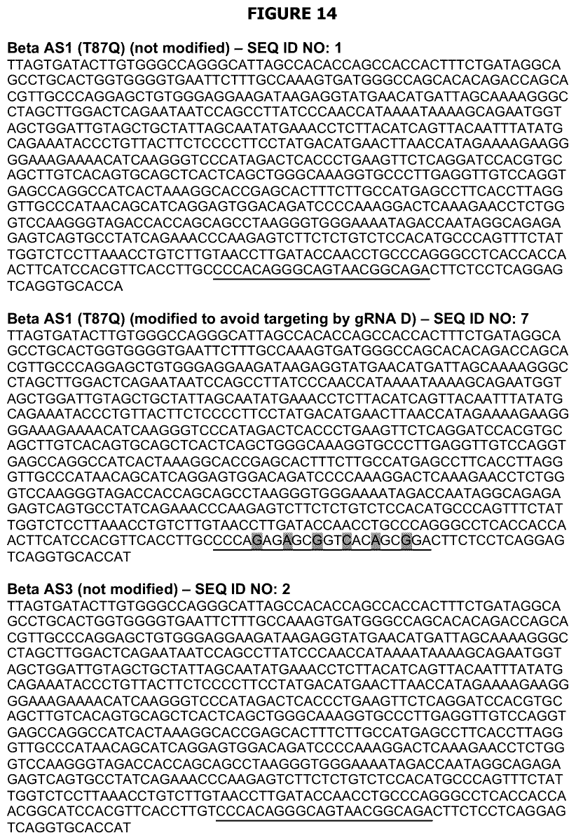

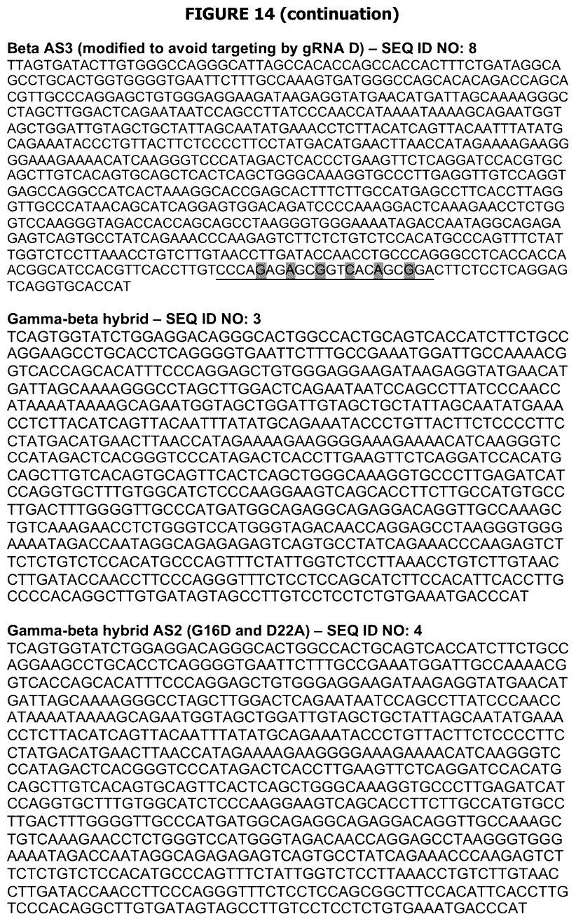

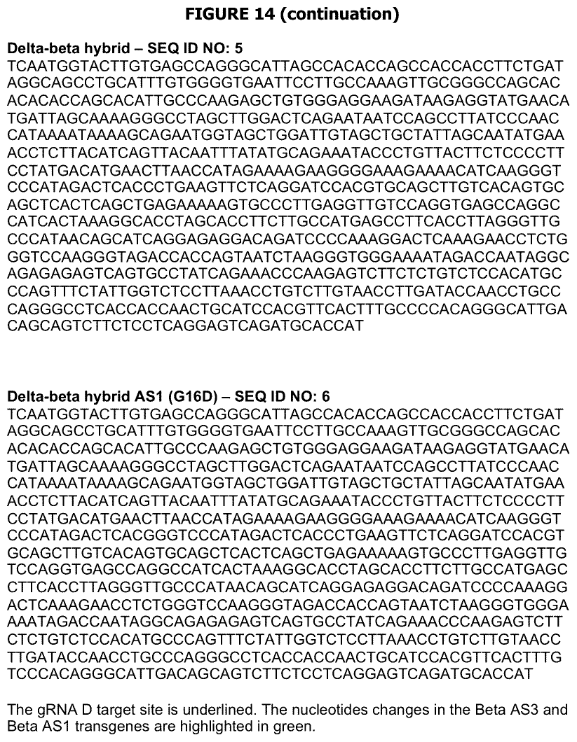

This process is illustrated in FIG. 13.

### Example 9: Evaluation of Transgene Expression, Genome Editing Efficiency and (i) Beta-Globin Down-Regulation (gRNA D) or (ii) Gamma-Globin Re-Activation (gRNA-13 bp-Del and gRNA-BCL11Aenhancer)

### Protocols

### Lentiviral Vectors Used

LV.GLOBE-AS3modified (LV.GLOBE.betaAS3-globin plasmid (SEQ ID NO: 45): lentiviral vector harboring only a Beta-AS3 transgene modified by inserting silent mutations in the sequence of exon 1 targeted by gRNA-D (AS3modified transgene), does not express gRNAD

LV.GLOBE-AS3modified.gRNAD (LV.GLOBE-AS3modified.gRNAD, SEQ ID NO: 94): lentiviral vector expressing AS3modified transgene and optimized gRNA D.

LV.GLOBE-AS3modified.gRNA-luciferase (SEQ ID NO: 93): lentiviral vector expressing AS3modified transgene and optimized gRNA targeting the luciferase gene, which is not present in the human genome.

LV.GLOBE-AS3modified.gRNA-BCL11Aenhancer (SEQ ID NO: 75): lentiviral vector expressing AS3modified transgene and optimized BCL11A gRNA (5′-CACAGGCTCCAGGAAGGGTT-3′-SEQ ID NO: 74) targeting the intronic erythroid-specific enhancer of BCL11A gene. To evaluate the editing efficiency of BCL11A gRNA by TIDE the following primers were used:

BCL11A-TIDE FORWARD:
(SEQ ID NO: 77)
5′-TGGACAGCCCGACAGATGAA-3′
BCL11A-TIDE REVERSE:
(SEQ ID NO: 78)
5′-AAAAGCGATACAGGGCTGGC-3′

LV.GLOBE-AS3modified.gRNA-13 bp-del (SEQ ID NO: 76): lentiviral vector expressing AS3modified transgene and optimized 13 bp-del gRNA (SEQ ID NO: 71) designed to reproduce the 13 bp small HPFH deletion within the promoters of HBG1 and HBG2 genes. To evaluate the editing efficiency of 13 bp-del gRNA by TIDE the following primers were used:

13 bp-del-TIDE FORWARD:
(SEQ ID NO: 79)
5′-AAAAACGGCTGACAAAAGAAGTCCTGGTAT-3′
13 bp-del-TIDE REVERSE:
(SEQ ID NO: 80)
5′-ATAACCTCAGACGTTCCAGAAGCGAGTGTG-3′

### Transduction of HUDEP-2 Cells

HUDEP-2 WT cells were transduced at MOI 50 with LVs LV.GLOBE-AS3modified.gRNAD (D, SEQ ID NO: 94), LV.GLOBE-AS3modified.gRNA-BCL11Aenhancer (BCL11A, SEQ ID NO: 75) and LV.GLOBE-AS3modified.gRNA-13 bp-del (13bpdel, SEQ ID NO: 76). Untransduced (UT) samples or cells transduced with LV.GLOBE-AS3modified (AS3, SEQ ID NO: 45) and LV.GLOBE-AS3modified.gRNA-luciferase (Luc) LVs were used as controls.

10 days after transduction, transduced cells were transfected using 4 μg GFP-Cas9 plasmid (pMJ920, Addgene plasmid #42234). After 18 hours plasmid-transfected Cas9-GFP+ cells (29%-45%, not shown) were sorted by FACS.

In parallel, an LVs LV.GLOBE-AS3modified.gRNAD-transduced sample was electroporated using 10 μg (60 pmol) of Cas9-GFP protein by using Nucleofector 4D (CA-137 program), achieving ≈90% of GFP+ Cas9-expressing cells (not shown).

Sorted plasmid-transfected and unsorted Cas9-protein-transfected D samples, as well as non-transduced and non-transfected cells (UT) and transduced but non-transfected samples used as controls were then differentiated in mature erythroblasts.

mRNAs Quantification

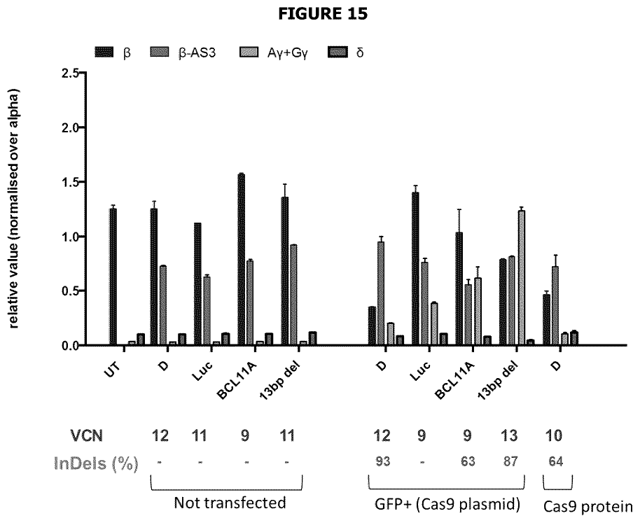

Globin mRNA expression in mature erythroblasts (day 9 of differentiation) is presented in FIG. 15.

Globin expression was evaluated by qRT-PCR in samples collected at day 9 of differentiation. Total RNA was extracted using RNeasy micro kit (QIAGEN) following manufacturer's instructions. Mature transcripts were reverse-transcribed using SuperScript First-Strand Synthesis System for RT-PCR (Invitrogen) with oligo(dT) primers. qRT-PCR was performed using SYBR green (Applied Biosystems).

Primers HBG1+HBG2 FORWARD: 5′-CCTGTCCTCTGCCTCTGCC-3′ (SEQ ID NO: 81) and HBG1+HBG2 REVERSE: 5′-GGATTGCCAAAACGGTCAC-3′ (SEQ ID NO: 82) were used to amplify the γ-globin transcripts. Primers HBB-AS3 FORWARD 5′-AAGGGCACCTTTGCCCAG-3′, (SEQ ID NO: 21) and HBB-AS3 REVERSE 5′-GCCACCACTTTCTGATAGGCAG-3′ (SEQ ID NO: 22) were used to amplify exclusively the beta AS3 globin transcripts. Primers HBB FORWARD: 5′-AAGGGCACCTTTGCCACA-3′, (SEQ ID NO: 81) and HBB REVERSE: 5′-gccaccactttctgataggcag-3′ (SEQ ID NO: 82) were used to amplify the endogenous β-globin transcripts. Primers HBD FORWARD: 5′-CAAGGGCACTTTTTCTCAG-3′ (SEQ ID NO: 85) and HBD REVERSE: 5′-AATTCCTTGCCAAAGTTGC-3′ (SEQ ID NO: 86) were used to amplify the δ-globin transcripts. Primers HBA1 F (5′-CGGTCAACTTCAAGCTCCTAA-3′, SEQ ID NO: 13) and HBA1 R (5′-ACAGAAGCCAGGAACTTGTC 3′, SEQ ID NO: 14) were used to amplify the alpha-globin transcripts. Endogenous beta-globin, AS3 beta-globin, gamma-globin and delta-globin results were normalized to alpha-globin.

BCL11A mRNA expression in undifferentiated (day0) HUDEP WT cells and in differentiated erythroblasts at different days of differentiation (day5, day7 and day9) was evaluated by qRT-PCR (as described above) in samples transduced with LV.GLOBE-AS3modified.gRNA-BCL11Aenhancer with or without transfection with Cas9-GFP plasmids followed by flow cytometry-based selection of GFP+ cells. Time-course analysis of the total BCL11A mRNA isoforms and of the BCL11A isoform XL, mainly involved in the regulation of gamma-globin expression, was performed by using qRT-PCR with the following primers:

BCL11A FORWARD:
(SEQ ID NO: 87)
5′-AACCCCAGCACTTAAGCAAA-3′
BCL11A REVERSE:
(SEQ ID NO: 88)
5′-GGAGGTCATGATCCCCTTCT-3′
BCL11AXL FORWARD:
(SEQ ID NO: 89)
5′-ATGCGAGCTGTGCAACTATG-3′
BCL11AXL REVERSE:
(SEQ ID NO: 90)
5′-GTAAACGTCCTTCCCCACCT-3′
GAPDH FORWARD:
(SEQ ID NO: 91)
5′-CTTCATTGACCTCAACTACATGGTTT-3′
GAPDH REVERSE:
(SEQ ID NO: 92)
5′-TGGGATTTCCATTGATGACAAG-3′

### HPLC Analyses of Globin Chains and Hemoglobin Tetramers

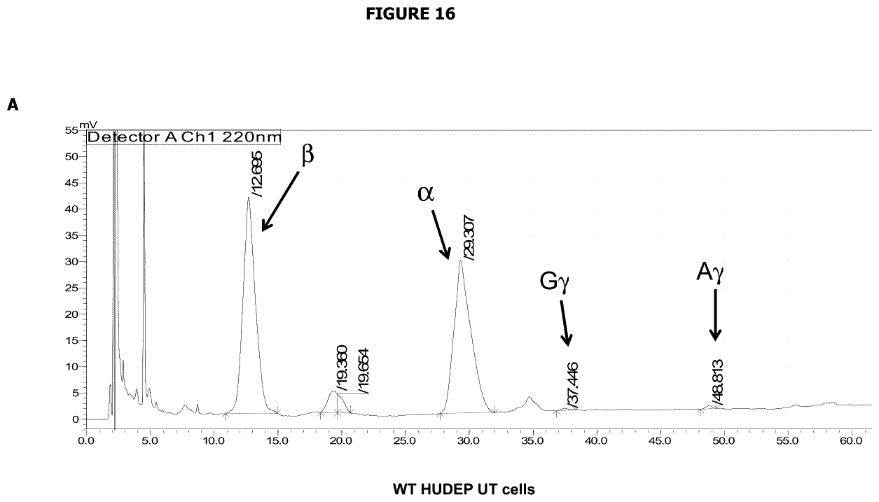

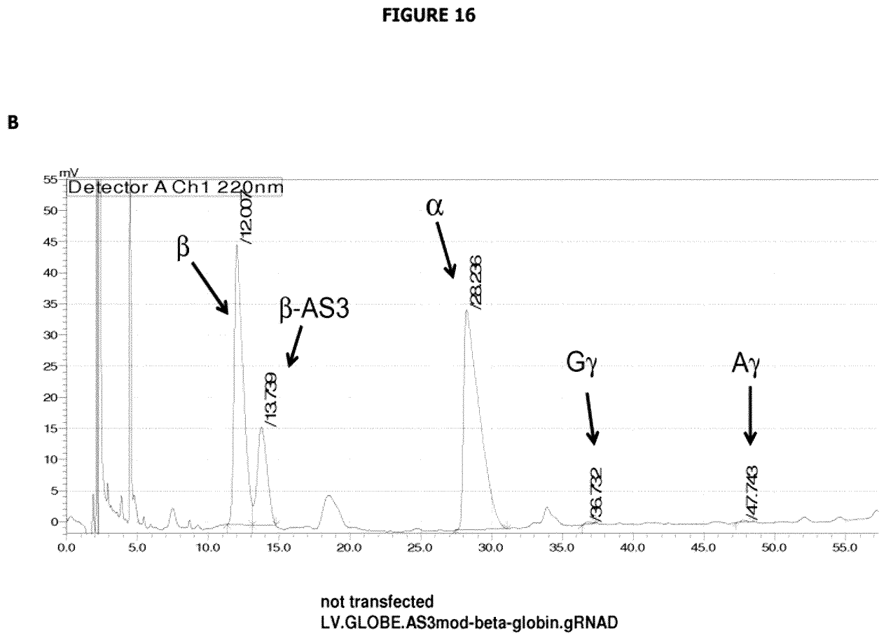

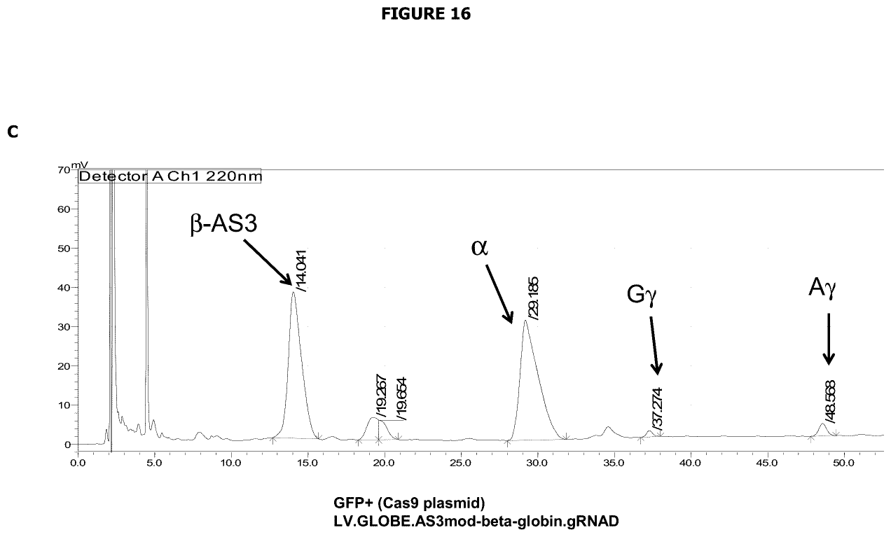

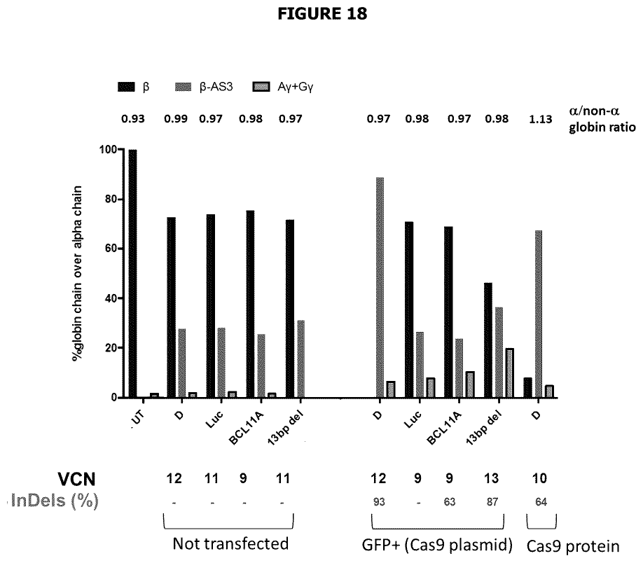

Globin chain profiles obtained using reverse phase HPLC in mature erythroblasts derived from control or genetically modified HUDEP cells (day 9 of differentiation) are presented in FIG. 16. Quantification of beta-like globin protein levels normalized to alpha-globin levels are shown in FIG. 18.

Briefly, reverse phase HPLC (RP-HPLC) analysis of globin chains was performed using a NexeraX2 SIL-30AC chromatograph (Shimadzu) and the LC Solution software. Globin chains from in vitro differentiated mature erythroblasts were separated by HPLC using a 250×4.6 mm, 3.6 μm Aeris Widepore column (Phenomenex). Samples were eluted with a gradient mixture of solution A (water/acetonitrile/trifluoroacetic acid, 95:5:0.1) and solution B (water/acetonitrile/trifluoroacetic acid, 5:95:0.1). The absorbance was measured at 220 nm.

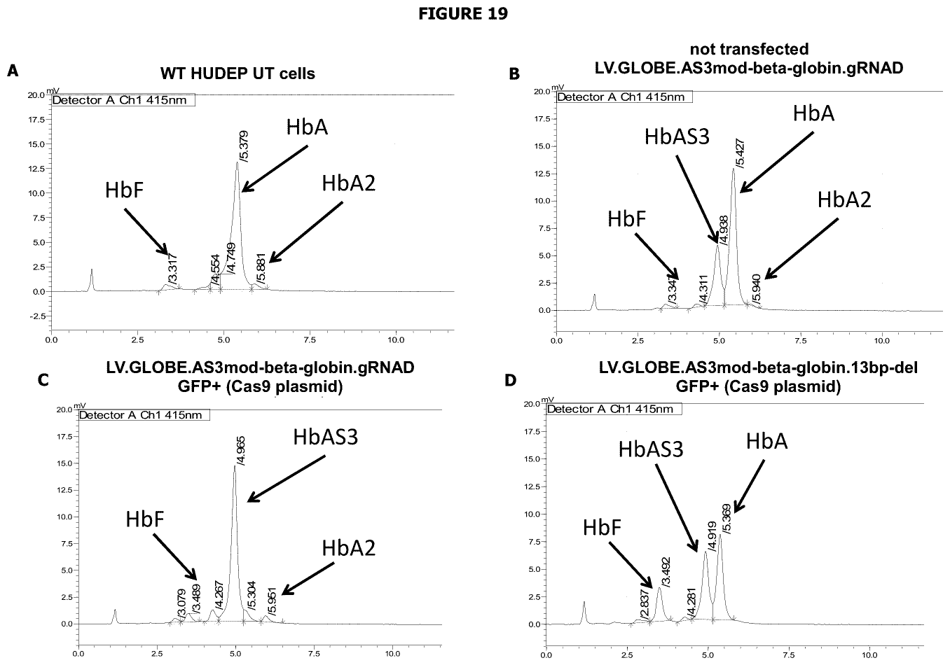

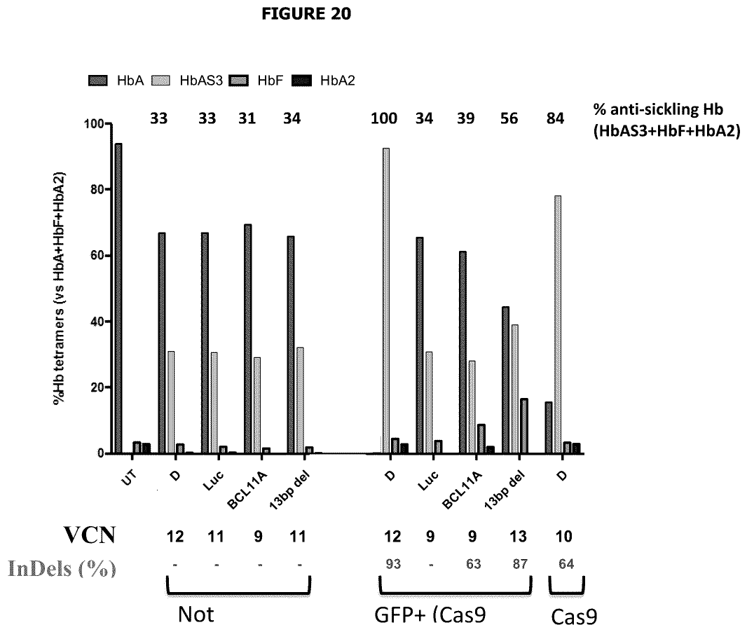

Hemoglobin profiles obtained using cation-exchange HPLC in mature erythroblasts derived from unmodified or genetically modified HUDEP cells (day 9 of differentiation) are presented in FIG. 19. Results of the quantification of each hemoglobin tetramer (HbA, HbAS3, HbF and HbA2) were reported as percentage over the total amount of hemoglobin tetramers and are shown in FIG. 20.

Analysis of hemoglobin tetramers was performed by cation-exchange HPLC using a NexeraX2 SIL-30AC chromatograph (Shimadzu) and the LC Solution software. Hemoglobin tetramers from mature erythroblasts were separated using a 2 cation-exchange column (PolyCAT A, PolyLC, Columbia, MD). Samples were eluted with a gradient mixture of solution A (20 mM bis Tris, 2 mM KCN, pH=6.5) and solution B (20 mM bis Tris, 2 mM KCN, 250 mM NaCl, pH=6.8). The absorbance was measured at 415 nm.

### Results:

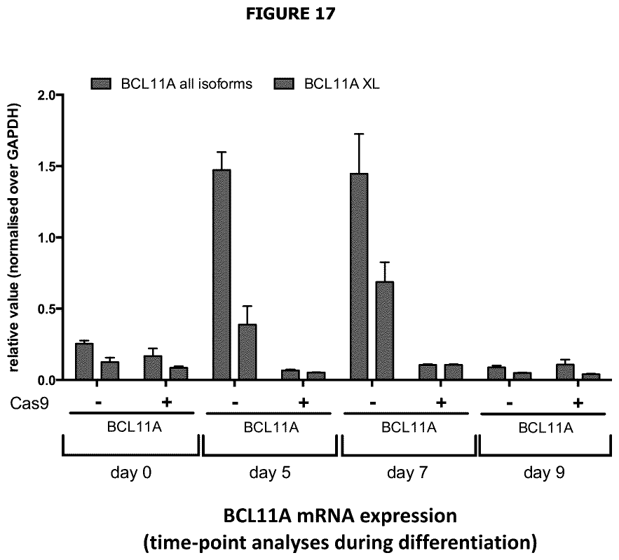

A) Globin (FIG. 15) and BCL11A mRNA Expression (FIG. 17)

### 1) Cells not Transfected (not Transfected)

AS3mod (not shown in the figures): higher expression level of β-AS3 associated with the higher VCN compared to other samples.

“Luc” transduced cells: Similar expression level of endogenous HBB mRNA compared to controls (UT) and lower expression of AS3 beta-globin mRNA transgene compared to AS3mod due to lower VCN (FIG. 15).

“D” transduced cells: no inactivation of endogenous β-globin gene (i.e. HBB), due to the absence of Cas9 delivery. Similar expression level of endogenous HBB mRNA compared to controls (UT and “luc”). “D” also expresses AS3 beta-globin mRNA transgene at similar level compared to control (“luc”) with similar VCN (FIG. 15).

“BCL11A” and “13 bp del” transduced cells: no inactivation of endogenous β-globin gene (i.e. HBB), because of the expression of gRNAs that do not target HBB. Similar expression level of endogenous HBB mRNA in the BCL11A and 13 bp del samples compared to controls (UT and “luc”). Similar levels of expression of AS3 beta-globin mRNA transgene for both BCL11A and 13 bp del samples in comparison with control (“luc”) with similar VCN (FIG. 15).

Note that BCL11A/BCL11AXL mRNA expression levels are increased over-time with a peak at days 5 and 7 of differentiation in non-transfected BCL11A sample (used as control in FIG. 17).
2) Cells transfected with GFP-Cas9 plasmid (GFP+(Cas9 plasmid)) or with Cas9-GFP protein (Cas9 protein)

AS3mod and Luc transduced cells: no genome editing in the exon 1 of endogenous HBB gene, as well as in the gamma-globin promoters or in the intronic enhancer of BCL11A gene, due to the absence of gRNAs in the LV vector (AS3mod) or the presence of a gRNA targeting the luciferase gene (Luc). Similar expression levels of endogenous beta-, AS3 beta-, gamma- and delta-globin chains compared to samples transduced with the same LV but «not transfected» with Cas9-GFP plasmid.

“D” transduced cells: down-regulation of endogenous β-globin gene expression in comparison with D «not transfected» sample and controls samples, due to the targeting of endogenous HBB gene by gRNA D and plasmid or protein delivery of Cas9. The expression of β-AS3 transgene and gamma-globin chains (Aγ+Gγ) tend to increase maybe as a consequence of HBB downregulation.

“BCL11A” and “13 bp del” transduced cells: an up-regulation of gamma-globin chains (Aγ+Gγ) expression is observed in comparison with “BCL11A” and “13 bp del” «not transfected» samples and controls, due to the disruption of the erythroid-specific BCL11A enhancer (BCL11A sample) or to the deletion of the 13-bp region in gamma-globin promoters (13 bp del sample) as a consequence of gRNA expression and plasmid delivery of Cas9. Indeed, the treatment with Cas9 strongly downregulated the expression of BCL11A, including XL isoform, in mature erythroblasts derived from Cas9-GFP+ BCL11A sample demonstrating that gRNA targeting the BCL11A enhancer is effective in decreasing BCL11A expression in erythroid cells and consequently implying a deregulation of γ-globin gene expression (see for example FIG. 15 or protein expression levels below). The 13 bp del sample showed reduced expression of the endogenous beta-globin gene. Similar expression levels of β-AS3- and delta-globin chains compared to samples transduced with the same LVs but «not transfected» with Cas9-GFP plasmid.

### B) Protein Expression

HPLC analyses showed a dramatic down-regulation of endogenous beta-globin expression (“β”) and HbA tetramers (FIGS. 18 and 20) and increased amounts of exogenous β-AS3-globin and HbAS3 tetramers (FIGS. 18 and 20) in mature erythroblasts derived from HUDEP-2 cells transduced with LV.AS3-beta-globin.gRNAD and transfected with Cas9-GFP plasmid or Cas9 protein (FIG. 16 panel C and FIGS. 18 and 20), when compared LV.AS3-beta-globin.gRNAD transduced but non-transfected cells (FIG. 16 panel B and FIGS. 18 and 20).

In particular mature erythroblasts derived from HUDEP-2 cells transduced with LV.AS3-beta-globin.gRNA-D and transfected with Cas9-GFP plasmid or Cas9 protein showed almost a complete knock-down of endogenous beta-globin chain expression (“β”) and HbA tetramers compensated by the expression of exogenous β-AS3-globin expression and HbAS3 tetramers as demonstrated by the alpha/not-alpha ratio that is similar in control samples (FIGS. 18 and 20). Genome editing at HBB target site and, as a consequence, the reduction in endogenous beta-globin chain/HbA and the increase in beta-globin AS3/HbAS3, is VCN-dependent (not shown) but significant even at low VCN (VCN=3).

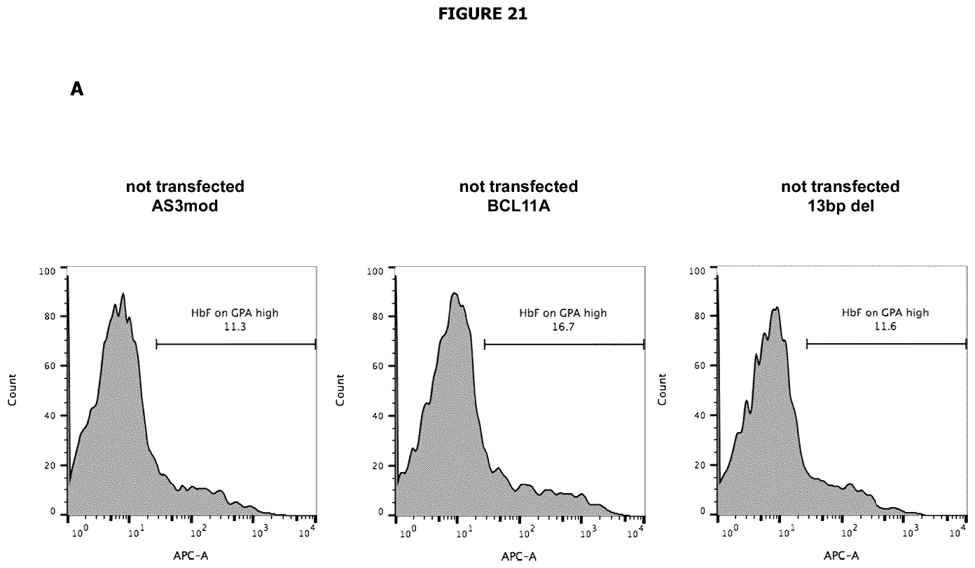

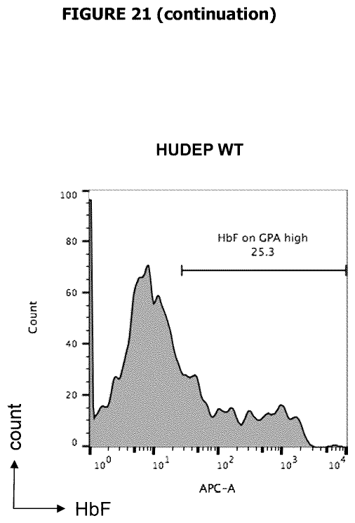

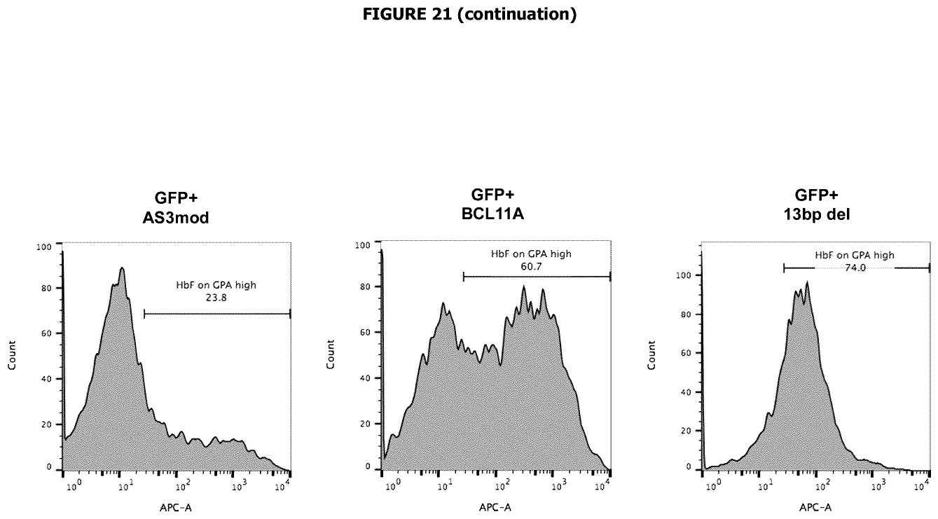

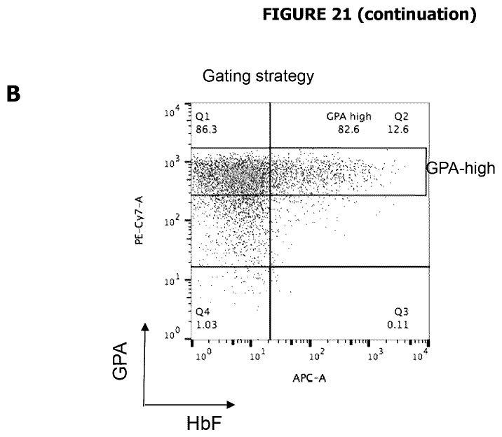

In mature erythroblasts derived from HUDEP-2 cells transduced with LV.AS3-beta-globin.gRNA-BCL11Aenhancer or LV.AS3-beta-globin.gRNA-13bpdel and transfected with Cas9-GFP plasmid, gamma-globin expression and HbF levels were significantly increased (FIGS. 18 and 20) compared to control samples and HbF expression pattern is close to be pan-cellular reaching 61% and 74% of F+ (HbF+) cells in mature erythroblasts derived from Cas9-expressing BCL11A and 13bpdel HUDEP-2, respectively (FIG. 21).

### Conclusions

Transgene expression at mRNA and protein levels (FIGS. 18 and 20) are correlated and are not impaired by gRNA expression and Cas9 delivery. Transgene expression is correlated with VCN at both mRNA (FIG. 15) and protein levels (FIGS. 18 and 20).

In Cas9-GFP+ D samples the knock-down of endogenous β-globin gene at mRNA level (FIG. 15) results in complete knock-out of endogenous β-globin protein expression (FIGS. 16 and 18) and absence of HbA tetramers (FIGS. 19 and 20). Hence a majority of anti-sickling tetramers (HbAS3) are observed in these cells.

The ratio between the expression of alpha-globin and non-alpha-globins (alpha/non-alpha ratio) is similar between all samples. The concomitant increase of anti-sickling globin expression (FIGS. 15-16), mainly AS3-β-globin (+60% in comparison with not-transfected D sample; FIGS. 16 and 19), compensates the observed robust endogenous β-globin downregulation. Hence, no modification in the balance between α- and other globin chain synthesis (FIGS. 18-19) is observed thereby avoiding generation of α-globin precipitates (FIGS. 19-20) which might be seen as a risk in the case of this therapeutic strategy.

Cas9 protein-mediated genome editing in “D” samples resulted in a clinically relevant switching between endogenous HbA tetramer (16%) and anti-sickling tetramers (HbAS3, HbF and HbA2; 84%) (FIGS. 19-20).

In mature erythroblasts derived from Cas9-GFP+ 13bpdel and BCL11A samples, a robust increase in γ-globin expression at both mRNA (FIG. 15) and protein (≈5 and ≈10 fold increase for 13bpdel and BCL11A, respectively; (FIG. 18)) levels in comparison with not-transfected 13bpdel and BCL11A samples was observed. Compared to matched non-transfected controls, in both 13bpdel and BCL11A samples an increased production of anti-sickling tetramers (+9% and +22% in 13bpdel and BCL11A, respectively; (FIGS. 19-20)) was observed, mainly associated with an enhanced generation of HbF tetramers. This finally resulted in ≈50% of HbA and ≈50% of HbAS3+HbF in 13bpdel sample, a condition resembling healthy heterozygous SCD carriers.

Relative amounts of HbA, HbA2, HbF and HbAS3 tetramers are shown in FIG. 20. Individuals with a level of of anti-sickling Hb above 50% are considered healthy (i.e. HbAS3+HbF+HbA2), which is the case for erythroblasts derived from HUDEP-2 cells transduced with D or 13bpdel and transfected with Cas9.

All together these results showed the effectiveness of the integrative system as set up by the inventors in:
inactivating mutant beta-globin gene involved in SCD pathophysiology when gRNA D is used; and
expressing HbAS3 and, when gRNA BCL11A or gRNA 13bpdel are used instead of gRNAD, increasing expression of γ-globin chains, resulting in the production of an amount of antisickling hemoglobin tetramers sufficient to correct sickle cell disease and avoid alpha-globin precipitations.

1. A genetically modified hematopoietic stem/progenitor cell (HSPC) obtainable by a method for modifying the genome of a HSPC, in vitro or ex vivo, comprising the steps of:
a) contacting a HSPC with a recombinant lentiviral vector to obtain a transduced HSPC, wherein the lentiviral vector is integrated into the genome of said HSPC; and
b) introducing into the transduced HSPC a catalytically active Cas9 or Cpf1 protein or a nucleotide sequence encoding a catalytically active Cas9 or Cpf1 protein, said catalytically active Cas9 or Cpf1 protein disrupts the expression and/or the function of a target gene when introduced or expressed into the transduced HSPC,
wherein the recombinant lentiviral vector comprises in its genome:
(i) a nucleotide sequence encoding a protein that has a therapeutic effect, said protein being selected from the group consisting of beta-globin, gamma-globin, delta-globin and variants thereof; and
(ii) a nucleotide sequence encoding a guide RNA (gRNA) that comprises a spacer adapted to bind to a target nucleotide sequence, said target nucleotide sequence is:
a. within the coding sequence or within a transcribed non-coding sequence of a target gene, said target gene is selected from beta-globin gene and BCL11A gene, or
b. within the promoter region of a target gene, said target gene is gamma-globin gene.

2. A genetically modified hematopoietic stem/progenitor cell (HSPC) obtainable by a method for preparing the genetically modified HSPC, in vitro or ex vivo, comprising the steps of:
a) contacting a HSPC with a recombinant lentiviral vector to obtain a transduced HSPC, wherein the lentiviral vector is integrated into the genome of said HSPC; and
b) introducing into the transduced HSPC a catalytically active Cas9 or Cpf1 protein or a nucleotide sequence encoding a catalytically active Cas9 or Cpf1 protein, said catalytically active Cas9 or Cpf1 protein disrupts the expression and/or the function of a target gene when introduced or expressed into the transduced HSPC,
wherein the recombinant lentiviral vector comprises in its genome:
(i) a nucleotide sequence encoding a protein that has a therapeutic effect, said protein being selected from the group consisting of beta-globin, gamma-globin, delta-globin and variants thereof; and
(ii) a nucleotide sequence encoding a guide RNA (gRNA) that comprises a spacer adapted to bind to a target nucleotide sequence, said target nucleotide sequence is:
a. within the coding sequence or within a transcribed non-coding sequence of a target gene, said target gene is selected from beta-globin gene and BCL11A gene, or
b. within the promoter region of a target gene, said target gene is gamma-globin gene.

3. The genetically modified HSPC according to claim 1, wherein a composition comprises the recombinant lentiviral vector or a plurality of said recombinant lentiviral vectors and the HSPC is contacted with the composition to obtain the transduced HSPC.

4. The genetically modified HSPC according to claim 2, wherein a composition comprises the recombinant lentiviral vector or a plurality of said recombinant lentiviral vectors and the HSPC is contacted with the composition to obtain the transduced HSPC.

5. A method for treating sickle cell disease (SCD) comprising administering the genetically modified HSPC according to claim 1.

6. A method for treating sickle cell disease (SCD) comprising administering the genetically modified HSPC according to claim 2.

7. A method for treating sickle cell disease (SCD) comprising administering the genetically modified HSPC according to claim 3.

8. A method for treating sickle cell disease (SCD) comprising administering the genetically modified HSPC according to claim 4.
